SEQUENCE LISTING (10 sequences)

SEQ ID NO: 1 - protein, 504 aa, Artificial Sequence
  CD40/CD40L+12mer linker
MVRLPLQCVLWGCLLTAVHPEHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKLGGGSGGGGSGGGPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRALVVIPIIFGILFAILLVLVFIKKVAKKPTNKAPHPKQEPQEINFPDDLPGSNTAAPVQETLHGCQPVTQEDGKESRISVQERQ

SEQ ID NO: 2 - protein, 506 aa, Artificial Sequence
  CD40/CD40L+14mer linker
MVRLPLQCVLWGCLLTAVHPEHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKLGGGGSGGGGSGGGGPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRALVVIPIIFGILFAILLVLVFIKKVAKKPTNKAPHPKQEPQEINFPDDLPGSNTAAPVQETLHGCQPVTQEDGKESRISVQERQ

SEQ ID NO: 3 - protein, 508 aa, Artificial Sequence
  CD40/CD40L+16mer linker
MVRLPLQCVLWGCLLTAVHPEHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKLGGGSGGGGSGGGGSGGPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRALVVIPIIFGILFAILLVLVFIKKVAKKPTNKAPHPKQEPQEINFPDDLPGSNTAAPVQETLHGCQPVTQEDGKESRISVQERQ

SEQ ID NO: 4 - protein, 510 aa, Artificial Sequence
  CD40/CD40L+18mer linker
MVRLPLQCVLWGCLLTAVHPEHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKLGSGGGGSGGGGSGGGGSGPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRALVVIPIIFGILFAILLVLVFIKKVAKKPTNKAPHPKQEPQEINFPDDLPGSNTAAPVQETLHGCQPVTQEDGKESRISVQERQ

SEQ ID NO: 5 - protein, 512 aa, Artificial Sequence
  CD40/CD40L+20mer linker
MVRLPLQCVLWGCLLTAVHPEHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKLGGSGGGGSGGGGSGGGGSGGPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRALVVIPIIFGILFAILLVLVFIKKVAKKPTNKAPHPKQEPQEINFPDDLPGSNTAAPVQETLHGCQPVTQEDGKESRISVQERQ

SEQ ID NO: 6 - protein, 515 aa, Artificial Sequence
  mouse_CD40/CD40L+12mer linker
MVSLPRLCALWGCLLTAVHLHRRLDKVEEEVNLHEDFVFIKKLKRCNKGEGSLSLLNCEEMRRQFEDLVKDITLNKEEKKENSFEMQRGDEDPQIAAHVVSEANSNAASVLQWAKKGYYTMKSNLVMLENGKQLTVKREGLYYVYTQVTFCSNREPSSQRPFIVGLWLKPSSGSERILLKAANTHSSSQLCEQQSVHLGGVFELQAGASVFVNVTEASQVIHRVGFSSFGLLKLGGGSGGGGSGGGGQCVTCSDKQYLHDGQCCDLCQPGSRLTSHCTALEKTQCHPCDSGEFSAQWNREIRCHQHRHCEPNQGLRVKKEGTAESDTVCTCKEGQHCTSKDCEACAQHTPCIPGFGVMEMATETTDTVCHPCPVGFFSNQSSLFEKCYPWTSCEDKNLEVLQKGTSQTNVICGLKSRMRALLVIPVVMGILITIFGVFLYIKKVVKKPKDNEILPPAARRQDPQEMEDYPGHNTAAPVQETLHGCQPVTQEDGKESRISVQERQVTDSIALRPLV

SEQ ID NO: 7 - protein, 517 aa, Artificial Sequence
  mouse_CD40/CD40L+14mer linker
MVSLPRLCALWGCLLTAVHLHRRLDKVEEEVNLHEDFVFIKKLKRCNKGEGSLSLLNCEEMRRQFEDLVKDITLNKEEKKENSFEMQRGDEDPQIAAHVVSEANSNAASVLQWAKKGYYTMKSNLVMLENGKQLTVKREGLYYVYTQVTFCSNREPSSQRPFIVGLWLKPSSGSERILLKAANTHSSSQLCEQQSVHLGGVFELQAGASVFVNVTEASQVIHRVGFSSFGLLKLGGGGSGGGGSGGGGGQCVTCSDKQYLHDGQCCDLCQPGSRLTSHCTALEKTQCHPCDSGEFSAQWNREIRCHQHRHCEPNQGLRVKKEGTAESDTVCTCKEGQHCTSKDCEACAQHTPCIPGFGVMEMATETTDTVCHPCPVGFFSNQSSLFEKCYPWTSCEDKNLEVLQKGTSQTNVICGLKSRMRALLVIPVVMGILITIFGVFLYIKKVVKKPKDNEILPPAARRQDPQEMEDYPGHNTAAPVQETLHGCQPVTQEDGKESRISVQERQVTDSIALRPLV

SEQ ID NO: 8 - protein, 519 aa, Artificial Sequence
  mouse_CD40/CD40L+16mer linker
MVSLPRLCALWGCLLTAVHLHRRLDKVEEEVNLHEDFVFIKKLKRCNKGEGSLSLLNCEEMRRQFEDLVKDITLNKEEKKENSFEMQRGDEDPQIAAHVVSEANSNAASVLQWAKKGYYTMKSNLVMLENGKQLTVKREGLYYVYTQVTFCSNREPSSQRPFIVGLWLKPSSGSERILLKAANTHSSSQLCEQQSVHLGGVFELQAGASVFVNVTEASQVIHRVGFSSFGLLKLGGGSGGGGSGGGGSGGGQCVTCSDKQYLHDGQCCDLCQPGSRLTSHCTALEKTQCHPCDSGEFSAQWNREIRCHQHRHCEPNQGLRVKKEGTAESDTVCTCKEGQHCTSKDCEACAQHTPCIPGFGVMEMATETTDTVCHPCPVGFFSNQSSLFEKCYPWTSCEDKNLEVLQKGTSQTNVICGLKSRMRALLVIPVVMGILITIFGVFLYIKKVVKKPKDNEILPPAARRQDPQEMEDYPGHNTAAPVQETLHGCQPVTQEDGKESRISVQERQVTDSIALRPLV

SEQ ID NO: 9 - protein, 521 aa, Artificial Sequence
  mouse_CD40/CD40L+18mer linker
MVSLPRLCALWGCLLTAVHLHRRLDKVEEEVNLHEDFVFIKKLKRCNKGEGSLSLLNCEEMRRQFEDLVKDITLNKEEKKENSFEMQRGDEDPQIAAHVVSEANSNAASVLQWAKKGYYTMKSNLVMLENGKQLTVKREGLYYVYTQVTFCSNREPSSQRPFIVGLWLKPSSGSERILLKAANTHSSSQLCEQQSVHLGGVFELQAGASVFVNVTEASQVIHRVGFSSFGLLKLGSGGGGSGGGGSGGGGSGGQCVTCSDKQYLHDGQCCDLCQPGSRLTSHCTALEKTQCHPCDSGEFSAQWNREIRCHQHRHCEPNQGLRVKKEGTAESDTVCTCKEGQHCTSKDCEACAQHTPCIPGFGVMEMATETTDTVCHPCPVGFFSNQSSLFEKCYPWTSCEDKNLEVLQKGTSQTNVICGLKSRMRALLVIPVVMGILITIFGVFLYIKKVVKKPKDNEILPPAARRQDPQEMEDYPGHNTAAPVQETLHGCQPVTQEDGKESRISVQERQVTDSIALRPLV

SEQ ID NO: 10 - protein, 523 aa, Artificial Sequence
  mouse_CD40/CD40L+20mer linker
MVSLPRLCALWGCLLTAVHLHRRLDKVEEEVNLHEDFVFIKKLKRCNKGEGSLSLLNCEEMRRQFEDLVKDITLNKEEKKENSFEMQRGDEDPQIAAHVVSEANSNAASVLQWAKKGYYTMKSNLVMLENGKQLTVKREGLYYVYTQVTFCSNREPSSQRPFIVGLWLKPSSGSERILLKAANTHSSSQLCEQQSVHLGGVFELQAGASVFVNVTEASQVIHRVGFSSFGLLKLGGSGGGGSGGGGSGGGGSGGGQCVTCSDKQYLHDGQCCDLCQPGSRLTSHCTALEKTQCHPCDSGEFSAQWNREIRCHQHRHCEPNQGLRVKKEGTAESDTVCTCKEGQHCTSKDCEACAQHTPCIPGFGVMEMATETTDTVCHPCPVGFFSNQSSLFEKCYPWTSCEDKNLEVLQKGTSQTNVICGLKSRMRALLVIPVVMGILITIFGVFLYIKKVVKKPKDNEILPPAARRQDPQEMEDYPGHNTAAPVQETLHGCQPVTQEDGKESRISVQERQVTDSIALRPLV

(19) United States
(12) Patent Application Publication
(10) Pub. No.: US 2021/0369825 A1
(43) Pub. Date: Dec. 2, 2021

(54) CD40 AND CD40L COMBO IN AN ADV VACCINE VEHICLE

(71) Applicant: NantCell, Inc., Culver City, CA (US)

(72) Inventors: Patrick Soon-Shiong, Culver City, CA (US); Kayvan Niazi, Culver City, CA (US)

(73) Assignees: NantCell, Inc., Culver City, CA (US); NantCell, Inc., Culver City, CA (US)

(21) Appl. No.: 17/277,997

(22) PCT Filed: Oct. 7, 2019
(86) PCT No.: PCT/US2019/055025

§ 371 Date: Mar. 19, 2021

Related U.S. Application Data
(60) Provisional application No. 62/742,167, filed on Oct. 5, 2018.
(60) Provisional application No. 62/755,217, filed on Nov. 2, 2018.

(51) Int. Cl. A61K 39/00; C07K 14/705; C07K 14/54; C12N 15/86

(52) U.S. Cl. CPC A61K 39/0011; C07K 14/70578; C07K 14/5415; A61K 2039/627; A61K 2039/53; A61K 2039/572; A61K 2039/575; C12N 15/86

(57) ABSTRACT

A cancer vaccine is provided including a recombinant nucleic acid encoding a self-activating chimeric signaling protein, and especially chimeric TNF family ligand-receptor proteins, and a tumor-associated antigen. In a preferred embodiment, the cancer vaccine may further include a nucleic acid segment encoding an IL-15 superagonist. In addition, the cancer vaccine can be co-administered with a genetically modified bacteria or yeast as an adjuvant to increase the payload expression of the cancer vaccine. Advantageously, cells expressing such combination of molecules will enhance immune reaction against tumor cells. Compositions and methods are presented that allow for an enhanced immune response against a vaccine composition, and particularly a recombinant adenoviral expression system that is used as a therapeutic agent. Most preferably, immune therapeutics are administered such that a protein or nucleotide are co-located with a therapeutic antigen, preferably via co-expression of the protein.

This application claims priority to U.S. provisional applications with the Ser. Nos. 62/742,167, filed Oct. 5, 2018, and 62/755,217, filed Nov. 2, 2018.

### SEQUENCE LISTING

The content of the ASCII text file of the sequence listing named Sequence listing ST25.TXT, which is 45 KB in size was created on Sep. 19, 2019 and electronically submitted via EFS-Web along with the present application, and is incorporated by reference in its entirety.

### FIELD

The present disclosure related to cancer vaccines that include a recombinant nucleic acid encoding a CD40/CD40L fusion protein and a tumor-associated antigen, as well as compositions and methods of improved neoepitope-based immune therapeutics. Certain particular disclosures relate to compositions comprising the nucleic acids and/or fusion molecules mentioned above, and methods of using these nucleic acids and/or fusion molecules to enhance immune response for cancer therapy.

### BACKGROUND

The following description includes information that may be useful in understanding the present disclosure. Nothing present herein constitutes an admission that any of the information provided herein is prior art or relevant to the presently claimed invention, or that any publication specifically or implicitly referenced is prior art.

All publications identified herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

TNF family member receptors such as CD40, 4-1BB, or OX40, and their respective ligands play a critical role in regulating cellular and humoral immunity. For example, 4-1BB signaling along with NK cell activation increases antibody dependent cellular cytotoxicity (ADCC) and interferon gamma (IFN-γ) secretion, while OX40 signaling is implicated in T cell activation and differentiation. In other examples, various immune cells express CD40, as do antigen presenting cells (APCs, e.g., dendritic cells, macrophages, and B cells). Among other roles, CD40L/CD40 is critical to activate and “license” dendritic cells to prime cytotoxic CD8+ T cells. Most typically, the CD40 ligand (CD40L) expressed on CD4+ helper T cells engages CD40 on APCs, inducing APC activation and maturation. CD40-licensed APCs induce activation and proliferation of antigen-specific CD8+ cytotoxic T cells. Notably, without CD40 signaling, CD8+ T cells and unlicensed APCs induce T cell anergy and trigger regulatory T cell formation, which is a mechanism by which tumors persist in a mammal despite presentation of otherwise antigenic peptides.

CD40 signaling can be effectively triggered using agonistic antibodies or soluble CD40L (e.g., Int Rev Immunol 2012, 31:246-66). However, such approach is limited by systemic toxicity (e.g., J Clin Oncol 2007, 25:876-83; Science 2012, 331:1612-16).

CD40 signaling efficacy depends on CD40 multimerization. A multi-trimeric fusion construct of CD40L and the gp100 tumor antigen activates dendritic cells and enhances survival in a B16-F10 melanoma DNA vaccine model (see e.g., Vaccine 2015 33(38):4798-806).

A chimeric polypeptide consisting of the CD40 signal transduction domain, fused to a 50-100 amino acid spacer, which was in turn fused to the CD40L binding and trimerization domain is reported in WO 00/63395.

Similarly, a chimeric polypeptide consisting of the CD40 signaling domain, fused to a type 2 receptor transmembrane domain, fused in turn to the CD40L binding and trimerization domain is reported in WO 02/36769.

Neither WO 00/63395 nor WO 02/36769 report finding a therapeutic effect in mice implanted with tumor cells transfected with these constructs.

A chimeric protein consisting of a CD40 cytoplasmic region fused to a FK506 ligand binding region and a myristoylation membrane targeting region is reported in U.S. Pat. No. 7,404,950.

A fusion protein with a multimeric ligand binding region and a CD40 portion lacking the extracellular domain is reported in U.S. Pat. No. 8,999,949.

While such constructs may provide some increased activity in vitro, they are prone to antigenicity when administered to mammals.

Therefore, while various manners of modulating TNF family member receptor/ligand signaling are known in the art, all or almost all of them suffer from one or more disadvantages. Thus, there is still a need for improved TNF family receptor/ligand signaling modulation.

Furthermore, immunotherapies targeting certain antigens common to specific cancers achieve remarkable responses in some patients. Unfortunately, many patients fail to respond to such immunotherapy, despite apparent expression of the same antigen. One possible reason for such failure could be that various immune effector cells may not have been present in sufficient quantities, or may have been exhausted. Moreover, intracellular antigen processing and HLA variability among patients may have led to insufficient antigen processing and/or display.

Some random mutations in tumor cells may give rise to unique tumor specific antigens (neoepitopes). Neoepitopes may provide a unique precision target for immunotherapy. Additionally, very small quantities of peptides can trigger cytolytic T-cell responses (e.g., Sykulev et al. (1996) Immunity, 4(6):565-71). Moreover, due to the relatively large number of mutations in many cancers, the number of possible targets is relatively high. In view of these findings, identifying cancer neoepitopes as therapeutic targets has attracted much attention. Unfortunately, current data suggest that almost all neoepitopes are unique to a patient and specific tumor and fail to provide any specific indication as to which neoepitope may be useful for an immunotherapeutic agent that is therapeutically effective.

However, even when neoepitopes are filtered for mutation type (e.g., to ascertain missense or nonsense mutation), for confirmed transcription of the mutated gene, for protein expression, and/or for specific HLA binding (as described in WO 2016/172722), a durable and therapeutically effective immune response may still be elusive. For example, immunity may be prevented by suppressive conditions in the tumor microenvironment. In addition, not all neoepitopes trigger an immune reaction with the same strength. Some neoepitopes may barely be immunogenic.

Even though multiple methods of neoeptiope identification and delivery to various cells are known, all of them suffer various disadvantages. Consequently, improved systems and methods for neoepitope selection and production to increase likelihood of a therapeutic response are desired.

### SUMMARY

Compositions, methods, and uses are disclosed herein of a recombinant nucleic acid encoding a chimeric protein comprising a TNF family member ligand and a TNF family member receptor, and encoding a tumor-associated antigen. Also disclosed are genetically modified immune cells including such nucleic acids. Also disclosed are methods of treating cancer using such recombinant nucleic acids and/or genetically modified immune cells. For example, a recombinant expression cassette is provided herein comprising a promoter operably coupled to a recombinant nucleic acid. The recombinant expression cassette can be an RNA, and/or a part of a viral expression vector. The recombinant expression cassette comprises a first nucleic acid segment encoding a chimeric protein having an extracellular portion of a TNF family member ligand coupled by a flexible linker to its corresponding TNF family member receptor, and a second nucleic acid segment encoding a tumor-associated antigen. Most typically, the extracellular portion of the TNF family member ligand is located N-terminally relative to the TNF family member receptorin the chimeric protein. In addition, preferably, the flexible linker has between 4 and 50 amino acids, and optionally comprises a (GnS)x sequence.

In certain embodiments the recombinant expression cassette comprises a third nucleic acid segment encoding a leader peptide that is coupled to the N-terminus of the extracellular portion of CD40L (CD40 ligand). In such embodiments, the extracellular portion of CD40L may be a human extracellular portion of CD40L. In some embodiments, the tumor-associated antigen is a selected from the group consisting of brachyury, MUC1, and CEA. In other embodiments, the tumor-associated antigen is a patient-and tumor-specific neoepitope.

In certain preferred embodiments, the first and second nucleic acid segments are placed in the same reading frame. Alternatively, the first and second nucleic acid segments can be coupled via an IRES or 2A sequence.

In certain embodiments, the recombinant expression cassette further may comprise a fourth nucleic acid segment encoding an immune stimulatory cytokine. In such embodiments, the immune stimulatory cytokine can be an IL-15 super agonist (ALT803) that is coupled with at least one of IL-7 and IL-21.

In certain embodiments, a genetically engineered virus can include the recombinant expression cassette described above.

In still other embodiments, a genetically modified immune cell may comprise a recombinant nucleic acid having first and second nucleic acid segments, and optionally the third and fourth nucleic acid segments. The first nucleic acid segment encodes a chimeric protein having an extracellular portion of a TNF family member ligand coupled by a flexible linker to its corresponding TNF family member receptor. The second nucleic acid segment encodes a tumor-associated antigen. Most typically, the extracellular portion of the TNF family member ligand is located in N-terminus and the TNF family member receptor is located in C-terminus of the chimeric protein. In addition, preferably, the flexible linker has between 4 and 50, or between 8 and 50, or even more amino acids, and optionally comprises a (GnS)x sequence.

The genetically modified immune cell may be derived from a dendritic cell, and more preferably, a dendritic cell of the patient (allogeneic dendritic cells). In such embodiments, the patient's own dendritic cells can be obtained from the patient's blood and expanded ex vivo before and/or after genetically modified with the recombinant nucleic acid.

Preferably, the recombinant nucleic acid comprises a third nucleic acid segment encoding a leader peptide that is coupled to the N-terminus of the extracellular portion of CD40L. In such embodiments, the CD40L extracellular portion is a human extracellular CD40L portion.

Also disclosed herein are methods of treating a patient having a tumor. Genetically engineered viruses can be administered to the patient in a dose and schedule effective to treat the tumor. Most typically, the genetically engineered viruses include recombinant nucleic acids having first and second nucleic acid segments. The first nucleic acid segment encodes a chimeric protein having an extracellular portion of a TNF family member ligand coupled by a flexible linker to its corresponding TNF family member receptor. The second nucleic acid segment encodes a tumor-associated antigen. Most typically, the extracellular portion of the TNF family member ligand is located N-terminally relative to the the TNF family member receptor in the chimeric protein. In addition, preferably, the flexible linker has between 4 and 50 amino acids, and optionally comprises a (GnS)x sequence.

In certain prefered embodiments, the recombinant nucleic acid comprises a third nucleic acid segment encoding a leader peptide coupled to the N-terminus of the extracellular portion of CD40L. In such embodiments, the CD40L extracellular portion may be a human CD40L extracellular portion. In some embodiments, the tumor-associated antigen is a selected from the group consisting of brachyury, MUC1, and CEA. In other embodiments, the tumor-associated antigen is a patient-and tumor-specific neoepitope.

In certain preferred embodiments, the first and second nucleic acid segments are placed in the same reading frame. Alternatively, the first and second nucleic acid segments can be coupled via an IRES sequence. Additionally, the recombinant nucleic acid further can further comprise a fourth nucleic acid segment encoding an immune stimulatory cytokine. In such embodiments, the immune stimulatory cytokine can be an IL-15 super agonist (ALT803), alone or coupled with at least one of IL-7 and IL-21.

Optionally, the method may further comprise administering a checkpoint inhibitor and/or an IL-15 super agonist (ALT803) to the patient, wherein the checkpoint inhibitor or ALT803 is coupled with at least one of IL-7 and IL-21. Further, the method may comprise co-administering a genetically modified bacteria or a genetically modified yeast as an adjuvant to the genetically engineered virus. In such embodiments, the genetically modified bacteria may express endotoxins at a level insufficient to induce a CD-14 mediated sepsis. In certain embodiments, the genetically modified yeast is GI-400 series recombinant immunotherapeutic yeast strain.

Use of the genetically engineered virus, the recombinant expression cassette, and/or the genetically modified immune cell are also disclosed herein for generating a pharmaceutical composition to treat a patient having a cancer or for treating a patient having a cancer.

In particularly preferred aspects, a CD40L-CD40 fusion protein is constructed and expressed in an APC wherein the fusion protein is capable of folding back on itself to transmit a CD40-mediated signal as if it were activated by a separate cell with a CD40L (e.g., CD4+ T cell). Similarly, in further contemplated aspects, 4-1BB ligand/4-1BB and Ox40L/Ox40 fusion proteins may be expressed in suitable immune competent cells.

A chimeric protein is described herein that includes in sequence from N- to C-terminus, a CD40L extracellular portion coupled to a flexible linker, to CD40. In certain embodiments, the chimeric protein also comprises a leader peptide coupled to the N-terminus of the CD40L extracellular portion.

In certain preferred embodiments, the CD40L extracellular portion is a human extracellular portion and the CD40 is a human CD40. In certain preferred embodiments, the flexible linker has between 4 and 25 or 8 and 50 amino acids (e.g., including a (GₙS)ₓ motif with n and x independently between 1 and 5). Most typically, CD40 lacks a signal sequence as compared to a full length sequence. In certain embodiments, the chimeric protein may have a sequence according to any one of SEQ ID NOs: 1-10.

Also disclosed herein are recombinant expression cassettes including a promoter operably coupled to a segment that encodes the chimeric protein as described above. The recombinant expression cassette may also include a second segment that encodes a cytokine and/or at least a portion of a peptide selected from the group consisting of a tumor associated antigen (TAA), a tumor specific antigen (TSA), a tumor specific neoepitope, and combinations thereof. The recombinant expression cassette may be an RNA, or may be part of a viral expression vector (which may or may not be encapsulated).

Recombinant cells are described herein that are transfected with a recombinant expression cassette as described herein. In certain embodiments, the cell is an APC (e.g., dendritic cell), and/or the cell is transiently transfected.

Also described herein are methods of enhancing an immune reaction against an antigen. These methods include transfecting an APC with a nucleic acid construct comprising a recombinant expression cassette as described herein, and contacting the transfected cell with the antigen or expressing the antigen in the transfected cell. Upon contact or expression, the transfected cell is then contacted with a CD4+ T cell and/or a CD8+ T cell.

By way of non-limiting example, tumor and patient specific neoepitopes, or at least a portion of a tumor associated antigen (TAA) or a tumor specific antigen (TSA) may be used as antigens for the above methods. Transfecting may be performed ex vivo, and contacting may be performed in vivo. Therefore, the reaction against the antigen may be an immune reaction against a tumor or against a virus (e.g., HIV) in an individual.

Methods of treating a tumor in an individual are also disclosed herein. These methods include transfecting an APC of the individual with a recombinant expression cassette as described herein, and contacting the transfected cell with a tumor antigen or expressing the tumor antigen in the transfected cell. Upon contact or expression, the transfected cell is then contacted with a CD4+ T cell and/or a CD8+ T cell of the individual.

As noted before, y betransfecting ma performed ex vivo, and contacting may be performed in vivo. Moreover, the tumor antigen may be a tumor and patient specific neoepitope, or at least a portion of a TAA or TSA. In preferred aspects, the APC is a dendritic cell, and the recombinant expression cassette is an mRNA or part of an adenovirus.

In certain embodiments, a chimeric protein and/or a recombinant cell as described herein may be used to treat a cancer or viral infection.

Various immune therapeutic compositions and methods are described herein. In particular, recombinant viral expression systems are described in which an adjuvant polypeptide is encoded along with multiple selected neoepitopes (typically in form of a rational-designed polypeptide with a trafficking signal) to increase antigen processing and presentation and maximize therapeutic effect.

Methods of generating expression vectors are described herein, along with expression vectors for enhanced immune therapy. These methods include constructing a recombinant nucleic acid having a sequence that encodes a polytope operably linked to a first promoter to drive expression of the polytope, and that further encodes an adjuvant polypeptide operably linked to a second promoter to drive expression of the adjuvant polypeptide. Most preferably, the polytope comprises a trafficking element that directs the polytope to a sub-cellular location (e.g., cytoplasm, recycling endosome, sorting endosome, lysosome, or extracellular membrane). Additionally or alternatively, the trafficking element can direct the polytope to the extracellular space. The polytope may also comprise a plurality of filtered neoepitope sequences.

In certain embodiments, the adjuvant polypeptide is calreticulin or HMGB1, or a portion of calreticulin or HMGB1with adjuvant activity. The first and/or second promoters can be constitutively active or inducible promoters (e.g., inducible by hypoxia, IFN-gamma, or IL-8). Suitable trafficking elements include but are not limited to cleavable ubiquitin, a non-cleavable ubiquitin, a CD1b leader sequence, a CD1a tail, a CD1c tail, and a LAMP 1-transmembrane sequence.

Most typically, filtered neoepitope sequences are filtered by comparing tumor versus matched normal sequences from the same patient. Sequences may also be filtered to have binding affinity to an MHC complex ≥200 nM. Moreover, the filtered neoepitope sequences may be arranged within the polytope such that the polytope has a likelihood of a presence and/or strength of hydrophobic sequences or signal peptides below a predetermined threshold.

In certain embodiments, the filtered neoepitope sequences bind to MHC-I and the trafficking element directs the polytope to the cytoplasm or proteasome. In certain embodiments, the filtered neoepitope sequences bind to MHC-I and the trafficking element directs the polytope to the recycling endosome, sorting endosome, or lysosome. In certain embodiments, the filtered neoepitope sequences bind to MHC-II and the trafficking element directs the polytope to the recycling endosome, sorting endosome, or lysosome. Additionally, the recombinant nucleic acid may further comprise a sequence encoding a second polytope, wherein the second polytope comprises a second trafficking element that directs the second polytope to a different sub-cellular location and wherein the second polytope comprises a second plurality of filtered neoepitope sequences. In such case, at least some of the plurality of filtered neoepitope sequences and some of the second plurality of filtered neoepitope sequences may be identical.

As disclosed herein, the recombinant nucleic acid may further comprise a sequence that encodes at least one of a co-stimulatory molecule (e.g., OX40L, 4-1BBL, CD80, CD86, CD30, CD40, CD30L, CD40L, ICOS-L, B7-H3, B7-H4, CD70, GITR-L, TIM-3, TIM-4, CD48, CD58, TL1A, ICAM-1, and LFA3), an immune stimulatory cytokine (e.g., IL-2, IL-12, IL-15, IL-15 super agonist (ALT803), IL-21, IPS1, and LMP1), and/or a protein that interferes with or down-regulates checkpoint inhibition (e.g., antibody or an antagonist of CTLA-4, PD-1, TIM1 receptor, 2B4, or CD160).

Suitable expression vectors include adenoviral expression vectors having E1 and E2b genes deleted, yeast expression vectors, and bacterial expression vectors. Recombinant viruses, yeast, and bacteria are described herein that comprise the expression vectors presented herein. Also described herein are pharmaceutical compositions comprising a recombinant virus, yeast, or bacterium carrying the recombinant expression vector. Use of an expression vector is also disclosed herein for treating cancer and/or for manufacturing a vaccine composition for treatment of cancer.

Methods of treating an individual are described herein. These methods include administering a vaccine composition that comprises an expression vector as presented herein, wherein the vaccine is administered under conditions effective to expose a dendritic cell of the individual to at least a portion of the polytope and at least a portion of the adjuvant polypeptide at the same time.

Alternatively or additionally, methods of improving immune response to cancer are described herein. These immune therapies in an individual include administering to the tumor of the individual a cancer vaccine composition, and co-administering to the tumor at substantially the same time (i.e., while the cancer vaccine composition is present in measurable quantities in the patient) an adjuvant polypeptide, ATP, or an ATP analog.

The cancer vaccine composition may comprise a recombinant adenovirus, a recombinant yeast, or a recombinant bacterium, and/or comprise or encodes a tumor neoepitope of the patient. By way of non-limiting example, one may administer the cancer vaccine composition directly to the tumor. Suitable adjuvant polypeptides include but are not limited to calreticulin or a portion with adjuvant activity thereof, or HMGB1 or a portion with adjuvant activity thereof. In certain embodiments, the adjuvant is a non-hydrolysable ATP analog. One may inject the adjuvant directly into the tumor.

Various objects, features, aspects and advantages of the technologies disclosed herein will become more apparent from the following detailed description and accompanying drawings.

### BRIEF DESCRIPTION OF DRAWINGS

FIG. 1 depicts several views of predicted structures of an exemplary fusion protein.

FIG. 2 depicts results for cells expressing exemplary fusion proteins.

FIG. 3 demonstrates that the constructs are operable in diverse species (murine).

FIG. 4 depicts secretion of IL-8 in selected cell lines.

FIG. 5 demonstrates that the constructs are operable across diverse species.

FIG. 6 depicts surface expression in 293T cells.

FIG. 7 depicts surface expression in B16F10 cells.

FIG. 8 compares 293T cells transfected with CD40 and subsequent stimulation with soluble CD40L versus a CD40-CD40L fusion.

FIG. 9 depicts 293T (human) and B16F10 (mouse) cytokine production from cells transfected with human/mouse constructs.

FIG. 10A illustrates an exemplary dimer for chimeras including hIL7 & IL21. 10B illustrates an exemplary dimer for chimeras including mIL7 & IL21. 10C illustrates an exemplary dimer for chimeras including hIL21. 10D illustrates an exemplary dimer for chimeras including hIL7. 10E illustrates an exemplary dimer for chimeras including hIL18 & IL12. 10F illustrates an exemplary dimer for chimeras including hIL18.

FIG. 11 is a schematic representation of various neoepitope arrangements.

FIG. 12 is an exemplary, partial schematic for selecting preferred neoepitope arrangements.

Prior Art FIG. 13 is a schematic illustration of cytoplasmic antigen processing and MHC-I presentation.

Prior Art FIG. 14 is a schematic illustration of lysosomal and endosomal antigen processing and MHC-II presentation.

FIG. 15 is a schematic illustration of a recombinant adenoviral expression cassette for a cancer vaccine.

### DETAILED DESCRIPTION

As disclosed herein, an immune response against a tumor cell can be modulated in a desired direction (i.e., enhanced or dampened) by interference with CD40 signaling in APCs. Immune response against a tumor cell can be significantly enhanced by inducing APCs to express one or more TAA(s). Vaccine compositions that induce expression of a chimeric protein and a TAA in the APC can treat tumors expressing the TAA. Thus, recombinant expression cassettes are described herein that include nucleic acid sequences encoding TAAs and chimeric proteins that modulate CD40 signaling events.

As used herein, “tumor” refers to, and is interchangeable with one or more cancer cells, cancer tissues, malignant tumor cells, or malignant tumor tissue, that can be placed or found in one or more anatomical locations in a human body. As used herein, “bind” can be interchangeably used with “recognize” and/or “detect” to convey an interaction between two molecules with affinity (K_{D}) equal or less than 10⁻³M, 10⁻⁴M , 10⁻⁵M , 10⁻⁶M, or equal or less than 10⁻⁷M. As used herein, “provide” or “providing” refers to and includes any acts of manufacturing, generating, placing, enabling to use, or making ready to use.

### Chimeric Proteins (CD40/CD40L)

As used herein, “chimera” is interchangeable with “chimeric protein.” Chimeric proteins disclosed herein preferably include a TNF family ligand (preferably an extracellular portion of a ligand) and its corresponding TNF family receptor. These chimeras can mimic or induce signaling cascades in cells. Exemplary TNF family ligands and corresponding receptors include CD40/CD40L, 4-1BB/4-1BBL, and OX-40/OX-40L. As these proteins share common structural motifs and activation patterns, the CD40/CD40L embodiments and examples presented herein equally apply to 4-1BB/4-1BBL and OX-40/OX-40L.

A chimeric protein having an extracellular portion of a TNF family member ligand and its corresponding TNF family member receptor can self-activate to elicit signal transduction. For example, a chimeric protein having an extracellular domain of CD40L and CD40 can be a self-activating CD40 signaling protein that is capable of folding back on itself and transmit CD40− mediated signal into APCs as if it had been contacted by another cell expressing CD40L (e.g., CD4+ T cell). CD40 is a type-1 membrane protein with the N terminus outside of the cell. CD40 is a master switch (e.g., on dendritic cells), while CD40L (e.g., located on CD4+ T cells, etc.) is a type 2 membrane protein with the C terminus on the outside of the cell. CD40, like many other members of the TNF family, needs to trimerize to effect signaling. Trimerization happens by CD40 interacting with CD40L's trimerization domain. By coupling CD40 to its own CD40L (with trimerization domain) via a linker, one can exploit this activation requirement to induce singalling.

Consequently, CD40/CD40L chimeras expressed in APCs necessarily trimerize and effect signaling without the need for another cell (typically a CD4+ T cell) to deliver the CD40L. Most preferably, the APC will also express or be exposed to an antigen of choice, and therefore present a portion of the antigen on the MHC system. Such APC enhance immune response, even in the absence of CD4+ T cells, which is significant in infections with pathogens that destroy or reduce CD4+ T cells (e.g., HIV). Immune reactions can be enhanced or down-regulated in a tailored antigen specific fashion by co-presentation of the chimeric protein with at least a portion of the antigen on MHC. For immune stimulation against a specific antigen, the chimera can trimerize. Conversely, for immune down-regulation, chimera trimerization can be reduced or inhibited.

Such constructs are particularly relevant to vaccines and other immune stimulating compositions (especially cancer vaccines) where the trimerization concept is transposed onto other TNF family members like 4-1BB, OX40, etc. to activate cells through gene expression. Therefore, systems and methods as described above are also suitable for use beyond APCs (e.g., for use with NK cells and derivatives (e.g., NK-92, aNK, haNK, tank, etc.), T cells and derivatives (e.g., CAR-T, TCR-T, TIL-T, etc.), B cells, etc.).

For example, all CD40 variants are suitable for use herein. However, particularly suitable CD40 variants include human and other mammalian CD40s. Numerous such sequences are known (see e.g., uniprot sequence database), and all are suitable for use herein. In certain non-limiting embodiments the CD40 signal peptide is removed and replaced with an upstream portion that includes a linker and the CD40L portion. For activating chimeric constructs, CD40 will typically retain its intracellular activation domain. On the other hand, where down-regulation is desired, the CD40 will have an intracellular truncation lacking a (functional) activation domain.

Most typically, the particular CD40 will match the APC species (e.g., human CD40 for human APC). Numerous modifications may be implemented to achieve a desired purpose. For example, the intracellular activation domain may be present in multiple copies, or be partially deleted, or entirely deleted. In other examples, one or more amino acids may be added as a tag for identification via immunohistochemistry. In still further examples, one or more amino acids may be exchanged (especially at the N-terminus) to increase half life. In less preferred aspects, the CD40 transmembrane domain may be replaced with another transmembrane domain.

CD40L sequences may vary considerably. All CD40L variants are suitable for use herein. However and as already noted above, human and other mammalian CD40Ls are particularly suitable. Numerous such sequences are known (see e.g., uniprot sequence database), and all of these are suitable for use herein. In certain non-limiting embodiments the CD40L will include its native signal peptide, however, other signal peptides may also be included or substituted. CD40L should retain its trimerization domain for activating chimeric constructs,. On the other hand, where down-regulation is desired, the CD40L may have a truncated trimerization domain or some other sufficient steric hindrance to disrupt trimerization.

Most typically, CD40L will be selected to match APC species (e.g., human CD40 for human APC, etc.). Numerous modifications may be implemented to achieve a desired purpose. For example, the trimerization domain may be optimized to increase affinity, or be partially or entirely deleted. In still further examples, one or more amino acids may be exchanged (especially at the N-terminus) to increase half life.

Suitable linkers typically enabled sufficient mobility between the CD40 and CD40L portions to permit all selective binding. Especially for activating chimeric molecules, the linker will be a polypeptide that has between 4 and 60 amino acids, with low or no immunogenicity. Suitable linkers include GS-type linkers with between 8 and 50, or between 4 and 25, and most preferably between 15 and 17 amino acids. There are numerous alternative linkers known (see e.g., Adv Drug Deliv Rev 2013 65(10):1357-69), and all of them are suitable for use herein.

### Expression Cassettes

Recombinant expression cassettes encoding the chimeric proteins described above can include a first nucleic acid segment encoding CD40L portion (an extracellular domain of CD40L and optionally a leader peptide coupled to the N-terminus of the extracellular domain of CD40L), the linker, and the CD40 portion in a single reading frame, so that the CD40L portion, the linker, and the CD40 portion can be encoded in a single polypeptide. Exemplary chimeric constructs are shown in SEQ ID NOs:1-10. Where the leader peptide is to be coupled with the extracellular domain of CD40L, the nucleic acid segment encoding the leader peptide can be in placed in the same reading frame with the segment encoding the CD40L extracellular domain, with or without a linker in between. Fusion proteins may include intervening sequences (e.g., 2A sequences) or may be direct fusions. Expression cassettes include a promoter (constitutive or inducible) to drivee expression of the sequences encoding the chimeric proteins. As the chimeric protein has a transmembrane portion, the chimera will typically have a signal sequence (optionally cleavable) to direct the chimera to the cell surface.

### Tumor-Associated Antigens

Recombinant expression cassettes as described herein frequently also include a second nucleic acid segment encoding a TAA such as MUC1, CEA, brachyury, RAS (e.g., a mutated RAS (e.g., RAS with G12V, Q61R and/or Q61L mutations, etc.), a tumor-specific antigen such as PSA, PSMA, HER2, or tumor- and patient-specific neoantigen or neoepitope, which can be identified from the patient's omics data. As used herein, “neoepitope” conveys expressed random mutations in a tumor cell that constitute a unique, tumor specific antigen. Neoepitopes may be identified by considering the type (e.g., deletion, insertion, transversion, transition, translocation, etc.) and impact of the mutation (e.g., non-sense, missense, frame shift, etc.), which may as such serve as a content filter through which silent and other non-relevant (e.g., non-expressed) mutations are eliminated. Neoepitope sequences can be defined as sequence stretches with relatively short length (e.g., 8-12 mers or 14-20 mers) wherein such stretches include the change(s) in the amino acid sequences. Most typically, but not necessarily, the changed amino acid will be at or near the central amino acid position. For example, a typical neoepitope may have the structure of A₄-N-A₄, or A₃-N-A₅, or A₂-N-A₇, or A₅-N-A₃, or A₇-N-A₂, where A is a proteinogenic wild type or normal (i.e., from corresponding healthy tissue of the same patient) amino acid and N is a changed amino acid (relative to wild type or relative to matched normal). Therefore, the neoepitope sequences include sequence stretches with relatively short length (e.g., 5-30 mers, more typically 8-12 mers, or 14-20 mers) wherein such stretches include the change(s) in the amino acid sequences. Where desired, additional amino acids may be placed upstream or downstream of the changed amino acid, for example, to allow for additional antigen processing in various cellular compartments (e.g., proteasome, endosome, lysosome).

In some embodiments, the recombinant expression cassettes may include sequences encoding one or more TAA under separate promoters or in different reading frames, such that the TAAs are expressed as separate molecules. In other embodiments, the recombinant expression cassette may include one or more sequences encoding TAAs as a polytope. As used herein, “polytope” conveys a tandem array of two or more antigens expressed as a single polypeptide. Preferably, two or more human disease-related antigens are separated by linker or spacer peptides. Any suitable length and order of peptide sequence for the linker or the spacer can be used. However, the linker is preferably between 3 and 30 amino acids long, preferably between 5 and 20 amino acids, and more preferably between 5 and 15 amino acids. Glycine-rich sequences (e.g., gly-gly-ser-gly-gly, etc.) are preferred to provide flexibility of the polytope between two antigens. The second nucleic acid segment may further include a trafficking signal to direct the tumor-associated antigen, tumor-specific antigen, neoepitope, and/or polytope to at least one of MHC-I and/or MHC-II complex, more preferably at least to the MHC-II complex.

In some embodiments, the first and second nucleic acid segments lie in the same reading frame, preferably downstream of the same promoter, such that the chimeric protein and the tumor associated antigen can be expressed concurrently. In other embodiments, an internal ribosome entry site (IRES) sequence separates the first and second nucleic acid segments, such that translation of the first and second nucleic acid segments initiates concurrently. Alternatively, the sequences may also include an intervening sequence portion (e.g., 2A sequence).

### Additional Molecules Encoded by the Recombinant Expression Cassette

Additionally, the recombinant expression cassette may further comprise a third nucleic acid segment encoding one or more co-stimulatory molecules and/or cytokines to modulate immune response in the tumor microenvironment. Suitable co-stimulatory molecules include B7.1 (CD80), B7.2 (CD86), CD30L, CD40, CD40L, CD48, CD70, CD112, CD155, ICOS-L, 4-1BB, GITR-L, LIGHT, TIM3, TIM4, ICAM-1, LFA3 (CD58), and members of the SLAM family. Suitable cytokines include immune stimulatory cytokines (e.g., IL-2, IL-15, IL-17, IL-21, etc.) for increasing immune response, or a down-regulating cytokine (e.g., IL-10, TGF-β, etc.) to dampen immune response. Alternatively, or additionally, the nucleic acid further may also include a sequence encoding at least one component of a SMAC (e.g., CD2, CD4, CD8, CD28, Lck, Fyn, LFA-1, CD43, and/or CD45 or their respective binding counterparts). In certain embodiments, the nucleic acid may additionally comprise a sequence encoding a STING pathway activator, such as a chimeric protein in which a transmembrane domain of LMP1 of EBV is fused to a signaling domain of IPS-1.

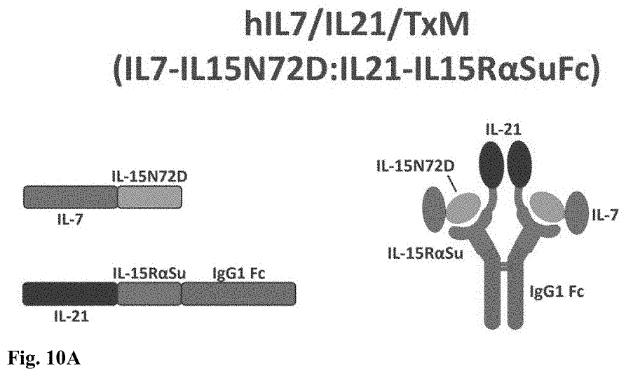

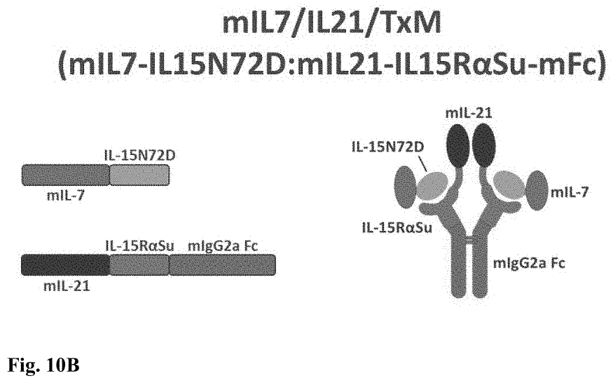

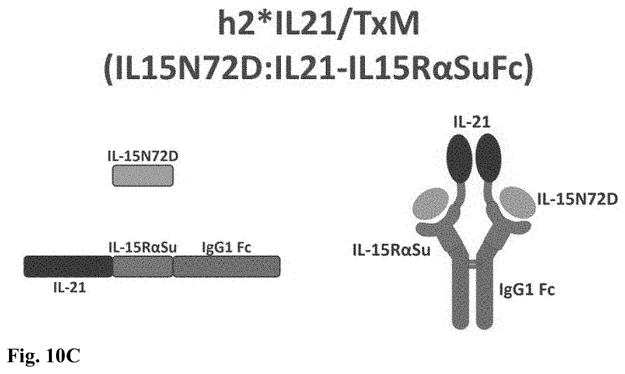

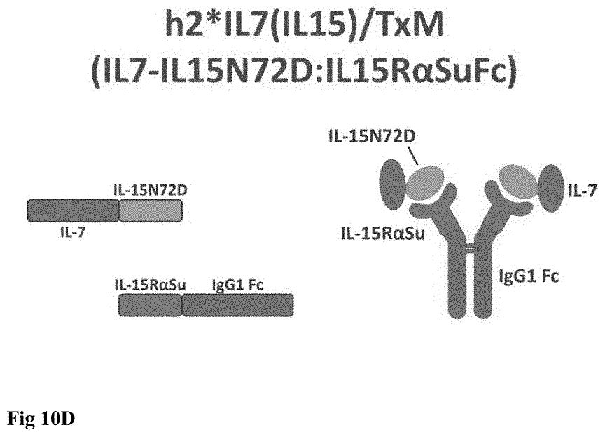

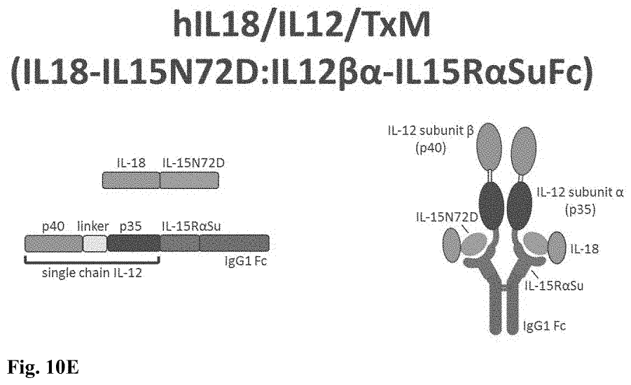

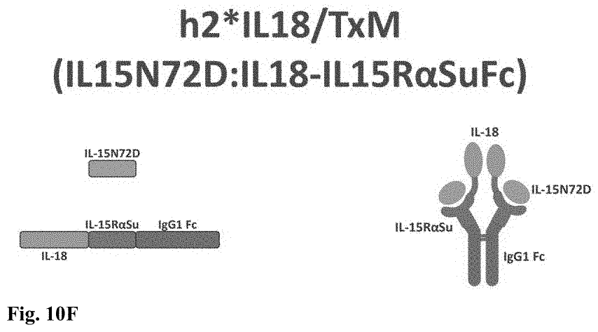

In one preferred embodiment, the cytokine is an IL-15 super agonist (IL-15N72D), and/or an IL-15 superagonist/IL-15Rα Sushi-Fc fusion complex (e.g., ALT-803) coupled with at least one of IL-7, IL-15, IL-18, IL-21, and IL-22, or preferably both IL-7 and IL-21. Any suitable variations of IL-15 superagonists are contemplated. Exemplary and preferred embodiments of IL-15 superagonists are shown in FIGS. 10A-10F.

### Expression Vectors

Most typically, the recombinant expression cassette is placed in an expression vector, such that the nucleic acid segment encoding the peptide can persist through cell divisions. For example, the recombinant expression cassette is a DNA/RNA fragment, and suitable DNA/RNA constructs may be linear or circular constructs configured as an expression vector. Thus, in one embodiment, a preferred expression vector includes a viral vector (e.g., nonreplicating recombinant adenovirus genome, optionally with a deleted or non-functional E1 and/or E2b gene, etc.). Such generated recombinant viruses may then be used—individually or in combination—as a therapeutic vaccine. Such vaccines are typically formulated as pharmaceutical compositions, e.g. sterile injectable compositions, with a virus titer between 10⁶ and 10¹³ virus particles, and more typically between 10⁹ and 10¹² virus particles per dosage unit.

In still further embodiments, the expression vector can be a bacterial vector that can be expressed in a genetically-engineered bacterium, which expresses endotoxins at a level low enough not to cause an endotoxic response in human cells and/or insufficient to induce a CD-14 mediated sepsis when introduced to the human body. Suitable bacteria include ClearColi® BL21(DE3) electrocompetent cells. This strain is BL21 with a genotype F—ompT hsdSB (rB- mB-) gal dcm lon λ(DE3 [lacI lacUV5-T7 gene 1 indl sam7 nin5]) msbA148 ΔgutQΔkdsD ΔlpxLΔlpxMΔpagPΔlpxPΔeptA. Several specific deletion mutations (ΔgutQ ΔkdsD ΔlpxL ΔlpxMΔpagPΔlpxPΔeptA) encode the modification of LPS to Lipid IV_{A}, while one additional compensating mutation (msbA148) enables the cells to maintain viability in the presence of IVA. These mutations delete the oligosaccharide chain from the LPS, more specifically, two of the six acyl chains. While electrocompetent BL21 bacteria are provided as an example, the genetically modified bacteria can be also chemically competent bacteria.

Alternatively or additionally, the expression vector can be a yeast vector that can be expressed in yeast. Preferred yeast include Saccharomyces cerevisiae (e.g., GI-400 series recombinant immunotherapeutic yeast strains, etc.).

The recombinant nucleic acids described herein need not be limited to viral, yeast, or bacterial expression vectors. Suitable vectors also include DNA vaccine vectors, linearized DNA, and mRNA, all of which can be transfected into suitable cells following protocols well known in the art.

### Virus Vaccine Formulation and Administration

Recombinant nucleic acids (or recombinant expression cassette) and/or the recombinant virus carrying the recombinant nucleic acids can be used to induce or generate antigen presenting cells (e.g., dendritic cells) in vivo or ex vivo. The chimeric proteins and TAAs produced can enhance anti-tumor immune response against cells expressing the TAA. One or more recombinant viruses including one or more nucleic acid segments encoding the chimeric protein and/or one or more tumor-associated antigen, cytokine, and/or co-stimulatory molecule can be administered to patient APCs in vivo. Such infected APCs express one or more TAAs, cytokines, and/or co-stimulatory molecules to stimulate immune response against the tumor cells.

For example, a genetically modified virus carrying the recombinant nucleic acid encoding the chimeric protein and/or one or more TAAs can be formulated in any pharmaceutically acceptable carrier (e.g., preferably formulated as a sterile injectable composition, etc.) to form a pharmaceutical composition. The sterile composition can be administered in any suitable methods. In some embodiments, where a cytokine (e.g., ALT-805) is to be expressed in the same cell, the recombinant nucleic acid further includes a nucleic acid encoding the cytokine. Additionally or alternatively, another recombinant virus (or bacteria or yeast) can be co-administered including a recombinant nucleic acid encoding the cytokine. Where two or more types of the recombinant virus are desired to infect the same antigen presenting cell, the two or more types of the recombinant virus can be formulated in a single pharmaceutical composition. However, the two or more types of the recombinant virus can also be formulated in two separate and distinct pharmaceutical compositions and administered to the patient concurrently or substantially concurrently (e.g., within an hour, within 2 hours, etc.)

Where the pharmaceutical composition includes the recombinant virus, the titer should be between 10⁴ and 10¹² virus particles per dosage unit. However, alternative formulations are also suitable for use herein, and all known routes and modes of administration. Where the pharmaceutical composition includes recombinant bacteria, the titer should be between 10² and 10³, between 10³ and 10⁴, or between 10⁴ and 10⁵ bacteria per dosage unit. Where the pharmaceutical composition includes recombinant yeast, the titer should be between 10² and 10³, between 10³ and 10⁴, or between 10⁴ and 10⁵ yeast per dosage unit.

As used herein, “administering” a virus, bacteria, or yeast formulation refers to both direct and indirect administration. Direct administration of the formulation is typically performed by a health care professional (e.g., physician, nurse, etc.). Indirect administration includes a step of providing or making available the formulation to the health care professional for direct administration (e.g., via injection, infusion, oral delivery, topical delivery, etc.).

In some embodiments, the virus, bacterial or yeast formulation is injected systemically, including subcutaneous, subdermal, or intravenous injection. In other embodiments, where the systemic injection may not be efficient (e.g., for brain tumors, etc.), the formulation may be injected into the tumor.

Dose and/or schedule of administration may vary depending on depending on the type of virus, bacteria, or yeast, type and prognosis of disease (e.g., tumor type, size, location), and health status of the patient (e.g., including age, gender, etc.). While it may vary, the dose and schedule may be selected and regulated so that the formulation has little significant toxic effect to normal host cells, yet sufficient to elicit an immune response. Thus, in a preferred embodiment, an optimal administration can be determined based on a predetermined threshold. For example, the predetermined threshold may be a predetermined local or systemic concentration of specific type of cytokine (e.g., IFN-γ, TNF-β, IL-2, IL-4, IL-10, etc.). Dose, route, and schedule are typically adjusted to have immune response-specific cytokines expressed at least 20%, at least 30%, at least 50%, at least 60%, at least 70% more at least locally or systemically.

For example, where the pharmaceutical composition includes recombinant virus, the dose is at least 10⁶ virus particles/day, or at least 10⁸ virus particles/day, or at least 10¹⁰ virus particles/day, or at least 10¹¹ virus particles/day. In some embodiments, a single dose of virus formulation can be administered at least once a day or twice a day (half dose per administration) for at least a day, at least 3 days, at least a week, at least 2 weeks, or at least a month. In other embodiments, the dose of the virus formulation can be gradually increased during the schedule, or gradually decreased during the schedule. In still other embodiments, several series of formulations can be administered, each separated by an interval (e.g., one administration each for 3 consecutive days and one administration each for another 3 consecutive days with an interval of 7 days, etc.).

In some embodiments, the formulation can be administered in two or more stages: e.g, a priming administration and a boost administration. The priming dose can be higher than the following boosts (e.g., at least 20% higher, preferably at least 40%, more preferably at least 60%, etc.). Alternatively, the priming dose can be lower than the following boosts. Additionally, where there is a plurality of boosts, each boost can have a different dose (e.g., increasing dose, decreasing dose, etc.).

### Cell-Based Composition & Administration

The patient's own APCs can be isolated from blood and transfected with recombinant nucleic acid encoding the chimeric protein and/or TAA. Isolated patient APCs can also be infected with recombinant virus, bacteria, or yeast including the recombinant nucleic acid. In some embodiments, MHC-matched heterologous APCs can be used with the patient's own APCs or instead of patient APCs. For example, patient dendritic cells (allogeneic dendritic cells) can be isolated and further expanded ex vivo with TNF-α, granulocyte-macrophage colony-stimulating factor (GM-CSF), or Interleukin (IL)-4. These dendritic cells (DCs) can then further be transfected with the recombinant nucleic acid or infected with a virus vaccine including the recombinant nucleic acid. Optionally, after transfection and/or infection, the infected and/or transfected DCs can be further expanded ex vivo to increase the DC population to be administered.

Transfected/infected DCs can be formulated in any pharmaceutically acceptable carrier (e.g., as a sterile injectable composition) with a cell titer of at least 10³ cells/mL, preferably at least 10⁵ cells/mL, more preferably at least 10⁶ cells/mL, and at least 1 mL, preferably at least 5 mL, more preferably and at least 20 mL per dosage unit. Alternative formulations are also suitable for use herein, and all known routes and modes of administration.

### Adjuvants to be Co-Administered

Viruses, bacteria, or yeast having the recombinant nucleic acid encoding the chimeric protein and/or one or more TAAs can be co-administered with one or more adjuvants and/or additional molecules to enhance effect. For example, expression of viral payloads (recombinant nucleic acid or cassette) and/or viral infection efficiency can be substantially increased by co-administering or co-exposing non-host cells as adjuvants. Suitable non-host cells may include cell belonging to a species other than the host cell species (e.g., human for a patient) or cells belonging to the host species, but exhibiting one or more stress or danger signals (e.g. cells exposed to chemotherapeutics, radiation, etc. to trigger NKG2DL expression, stress markers, pro-apoptotic markers, etc.). Most typically, however, suitable non-host cells will be bacteria and/or yeast, pathogenic or otherwise.

For example, suitable bacteria include those modified to have reduced LPS expression that would otherwise trigger an immune response and cause endotoxic responses. One exemplary bacteria strain with modified lipopolysaccharides includes ClearColi® BL21(DE3) electrocompetent cells. While electrocompetent BL21 bacteria is provided as an example, suitable genetically modified bacteria can also be chemically competent bacteria.

Alternatively, an inactive or weakened bovine tuberculosis (e.g., Bacillus Calmette-Guérin) can be an adjuvant. Further, the patient's own endosymbiotic bacteria can serve as a non-host cell. As used herein, the patient's “endosymbiotic bacteria” refers to bacteria residing in the patient's body without invoking any substantial immune response. Thus, the patient's endosymbiotic bacteria are normal flora of the patient. Endosymbiotic bacteria may include E. coli or Streptococcus that can be commonly found in human intestine or stomach. Endosymbiotic bacteria can be obtained from patient biopsy samples from intestine, stomach, oral mucosa, conjunctiva, or in fecal samples. The patient's endosymbiotic bacteria can then be cultured and transfected with nucleotides encoding human disease-related antigen(s). Bacterial non-host cells may also include pathogenic cells, including Bordetella pertussis and/or Mycobacterium bovis. Most typically, but not necessarily, the bacterial non-host cells will be killed before exposure to the host cells.

Numerous yeast strains are suitable for use herein. Typical, non-pathogenic yeasts include Saccharomyces cerevisiae, S. boulardi, Pichia pasteuris, Schizosaccharomyces pombe, Candida stellata, etc. Such yeast strains may be further genetically modified to reduce one or more adverse traits, and/or to express a recombinant proteins that further increase viral infectivity and/or expression. Suitable yeast strains are typically commercially available and can be modified via known protocols.

Without being bound by theory, one or more non-host cell components may act as a danger or damage signal, particularly where the host cells are immune competent cells. Therefore, not only non-host cells may be used, but also one or more immune stimulating portions thereof. Suitable portions include PAMP receptor ligands, DAMP receptor ligands, TLR ligands, CpG, ssDNA, and thapsigargin.

The exact ratio of non-host cells to host cells may vary considerably, depending on the type of host cell, the type of non-host cell (or component thereof), and the virus (or DNA/RNA). However, generally the ratio of host cell to non-host cell is 1:1 to about 1:100, or 1:10 to about 1:1,000, or 1:50 to about 1:5,000, or 1:100 to about 1:10,000, especially where immune competent cells are the host cells and bacterial cells are the non-host cells. Similarly, suitable ratios of host cell to non-host cell include 100:1 to about 10:1, or 1:1 to about 1:10, or 1:50 to about 1:5,00, or 1:100 to about 1:1,000, especially where the host cells are immune competant and the non-host cells are yeast.

Exposure of the host cell to the recombinant virus (or DNA/RNA) in the presence of the non-host cell may vary considerably. However, generally exposure times will be between several minutes and several hours, or between several hours and several days. For example, where the exposure is performed in vitro, exposure times may be between 10 minutes and 2 hours, or between 30 minutes and 4 hours, or between 60 minutes and 6 hours, or between 2 hours and 8 hours, or between 6 hours and 12 hours, or between 12 hours and 24 hours, or between 24 hours and 48 hours, or even longer. On the other hand, where the exposure is performed in vivo (e.g., via vaccine formulation), exposure times may be between 60 minutes and 6 hours, or between 6 hours and 12 hours, or between 12 hours and 24 hours, or between 24 hours and 48, or even longer. In such vaccination scheme, the host cell, the non-host cell, and the recombinant virus (or DNA or RNA) can be co-administered in the same formulation.

The viral, bacterial, or yeast formulation having the recombinant nucleic acid encoding the chimeric protein and/or one or more TAAs can be co-administered with one or more cytokines and/or a checkpoint inhibitor. Any cytokine capable of modulating the immune response (e.g., increase or decrease T cell activity, etc.) will serve. Most preferably, the cytokine is an IL-15 super agonist (IL-15N72D), and/or an IL-15 superagonist/IL-15RαSushi-Fc fusion complex (e.g., ALT-803) coupled with at least one of IL-7, IL-15, IL-18, IL-21, and IL-22, or preferably both IL-7 and IL-21. Exemplary cytokines are shown in FIGS. 10A-10F. Exemplary checkpoint inhibitors include antibodies or binding molecules to CTLA-4 (especially for CD8⁺ cells), PD-1 (especially for CD4⁺ cells), TIM1 receptor, 2B4, and CD160. Ipilimumab and nivolumab are suitable checkpoint inhibitors.

Without wishing to be bound by theory, co-administering recombinant virus and transfected/infected APCs to the patient will activate T cells against tumor cells expressing the TAAs in the tumor microenvironment by increasing the number of the TAA-presenting, pre-activated APCs (e.g., DCs) and by exposing such APCs to the helper T (Th) cells or other immune cells. Th cells interacting with such APCs may further activate the signaling cascade to generate more memory T cells and elicit immune response against tumor cells.

### EXAMPLES

Crystal structures of CD40, CD40L, CD40/CD40L complexes were used to determine a range of linker lengths that could tether CD40 & CD40L together while at the same time maintaining functionality. To that end, five linkers of varying length were modeled and recombinantly expressed. Several of the fusion proteins were tested.

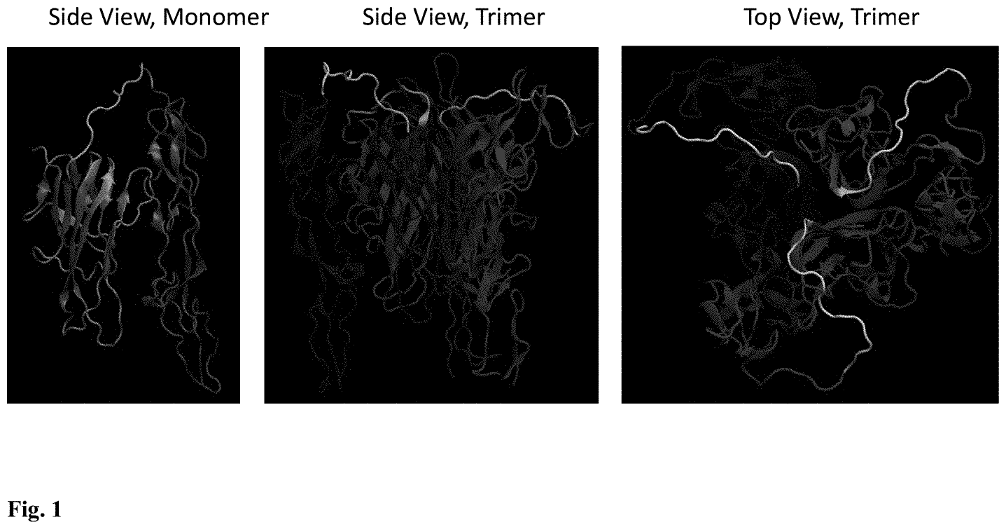

FIG. 1 depicts exemplary 16-mer linker models bearing fusion proteins. The left panel shows a predicted side view of the chimeric protein monomer. The middle panel depicts a predicted side view of the trimer. The right panel depicts a predicted top view of the trimer. As can be seen, the linker affords sufficient steric mobility to allow CD40L binding to CD40, and to allow trimerization.

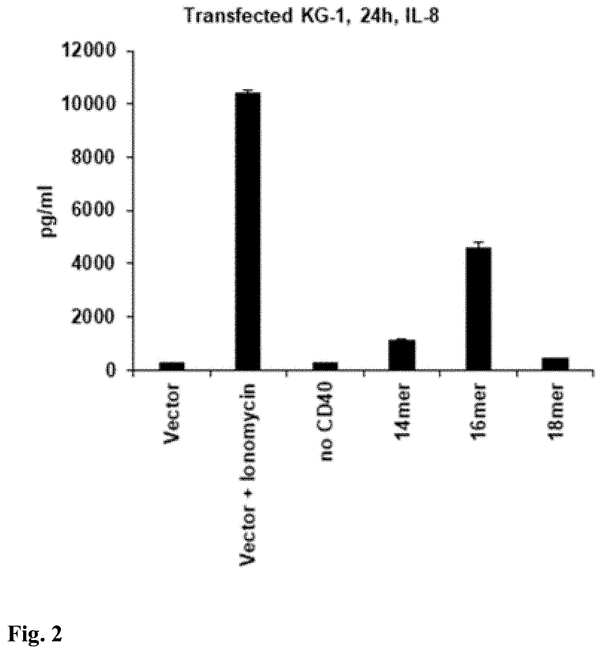

To determine whether these constructs would also stimulate immune competent cells, KG-1 cells (myeloid cell line) were transfected with constructs having different linker lengths. These cells transfect at about 30-50%. KG-1 cells were transfected via electroporation using BioRad Gene Pulser II, with 3 pulses (200 ohms, 25 μf, 0.26 kV), and cultured in growth medium (Iscove's Modified Dulbecco's Medium supplemented with 20% fetal bovine serum) for 16 hours. The transfected cells were washed to remove residual cytokines that may have resulted from the electroporation process, and cultured in fresh medium in a 96 well tissue culture plate at 20,000 cells per well. The cells were cultured for an additional 24 hours, and the supernatant was harvested. Cytokines levels in the supernantants were determined using Cytometric Bead Array specific for human IL-1β, MCP-1 and IL-8 according to the manufacturer's recommended protocol; however, only IL-8 demonstrated any changes. FIG. 2 shows IL-8 from human cells transiently transfected with CD40L-Linker-CD40 constructs with varying linker lengths. A linker length of about 16 amino acids was found to be most effective.

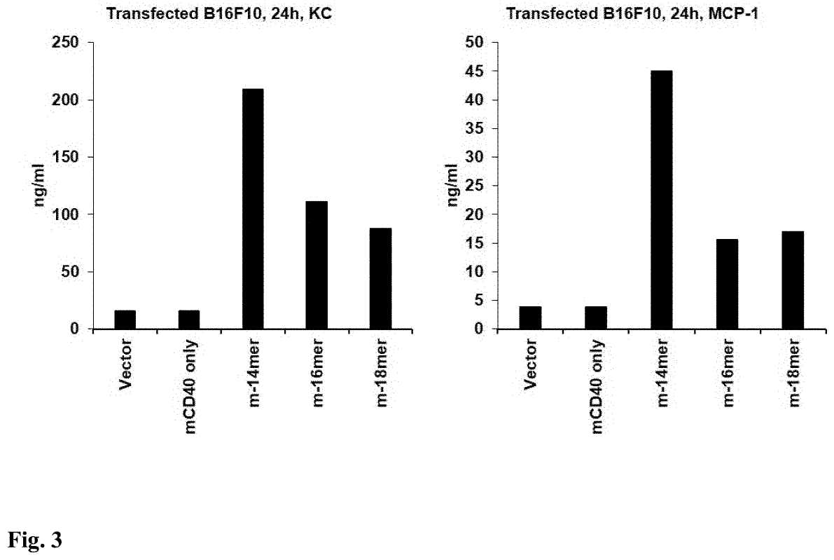

Mouse CD40L/CD40 fusion proteins: To determine whether the concept of self-ligating CD40/CD40L fusion constructs can be expanded to other species, a parallel set of constructs encoding the mouse versions of these proteins was produced and tested in the mouse B16F10 melanoma cell line for activity. B16F10 cells were transfected with the mouse CD40/CD40L fusion protein constructs using Lipofectamine 2000 according to the manufacturer's recommended protocol. The cells were rested for 18 hours, washed to remove residual cytokine and cultured in fresh growth medium (DMEM supplemented with 10% FBS) in a 96 well tissue culture plate at 50,000 cell per well for an additional 24 hours. Following incubation, supernatants were harvested and the levels of mouse IL-1β, MCP-1 and KC were determined using cytometric bead array, according to the manufacturer's recommended protocols. FIG. 3 shows that similar results were obtained in this parallel system indicating the system is likely to be expandable to other CD40 sequences and even other TNF family members. Some constructs triggered substantial activity in the transfected cells both (KC and MCP-1), indicating that a linker length of either 14 or 16 amino acids were most effective. The 18 amino acid linker did not elicit a response.

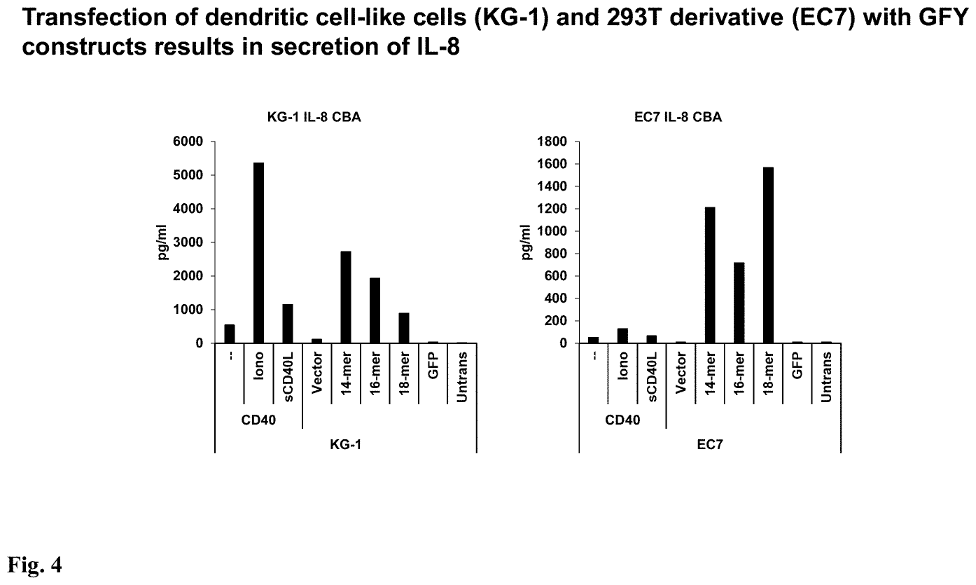

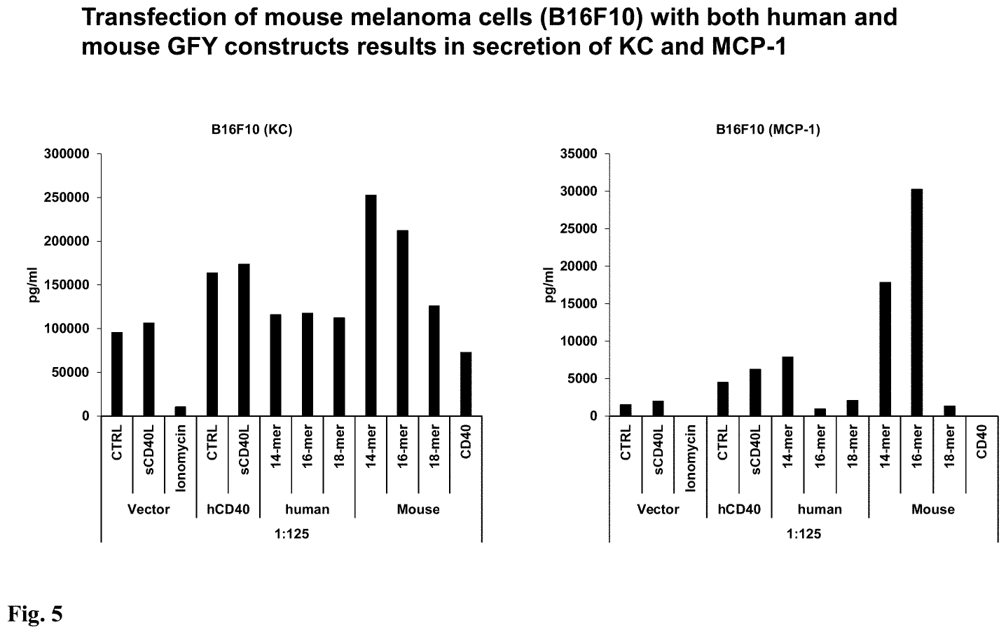

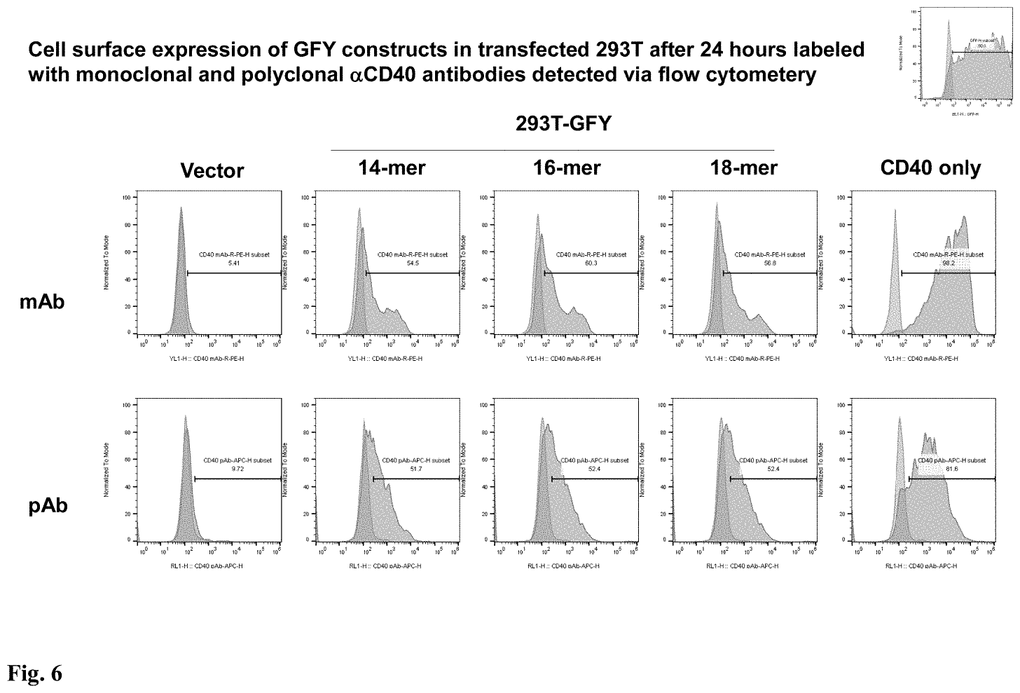

Using substantially same protocols as described above, dendritic cell-like (KG-1) and 293T derivative (EC7) cells transfected with the chimeric constructs to assay IL-8 secretion. FIG. 4 shows that both cell lines had significant IL-8 secretion with all variants tested. To further test whether the constructs could operate across species boundaries, mouse melanoma cells (B16F10) were transfected with both human and mouse constructs and assayed for secretion of KC and MCP-1. FIG. 5 shows KC and MCP-1 secretion even where the chimeric construct was not from the same species.

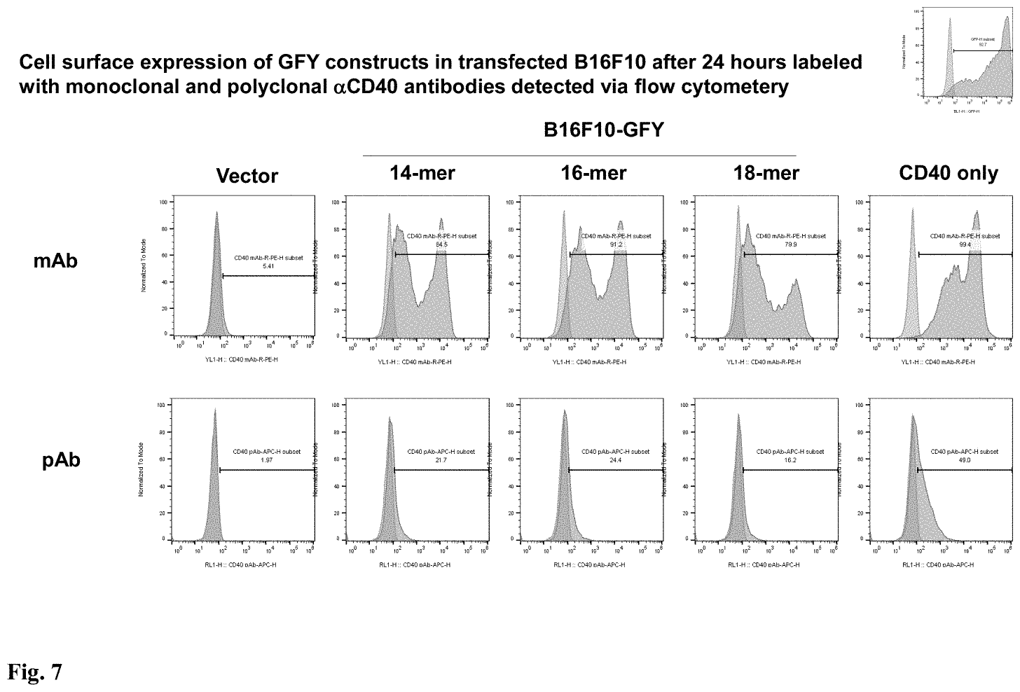

Human (293T) and murine (B16F10) cells were transfected and after 24 hours labeled with monoclonal or polyclonal antibodies. FIGS. 6 and 7 show that the CD40/CD40L constructs were expressed on the surfaces of both cell lines for all constructs.

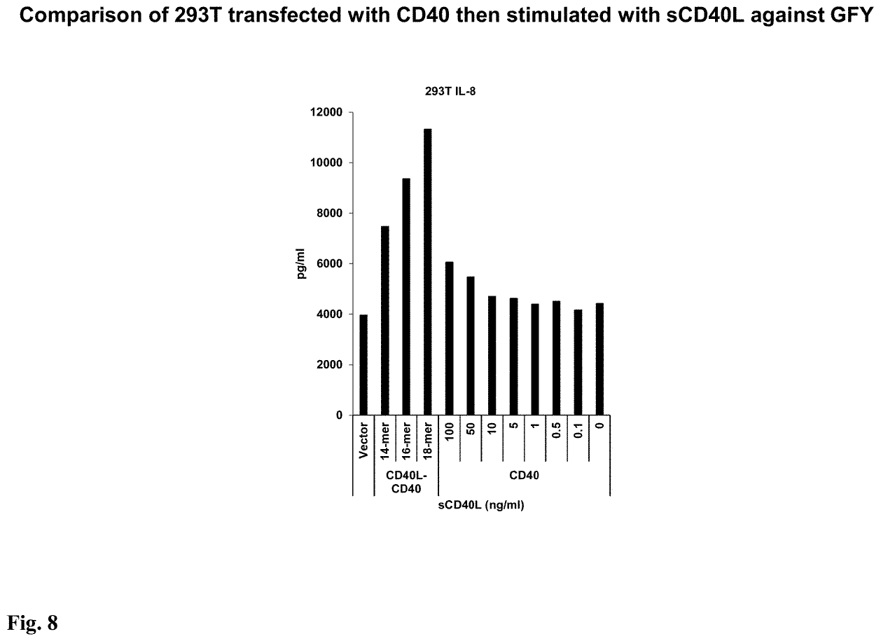

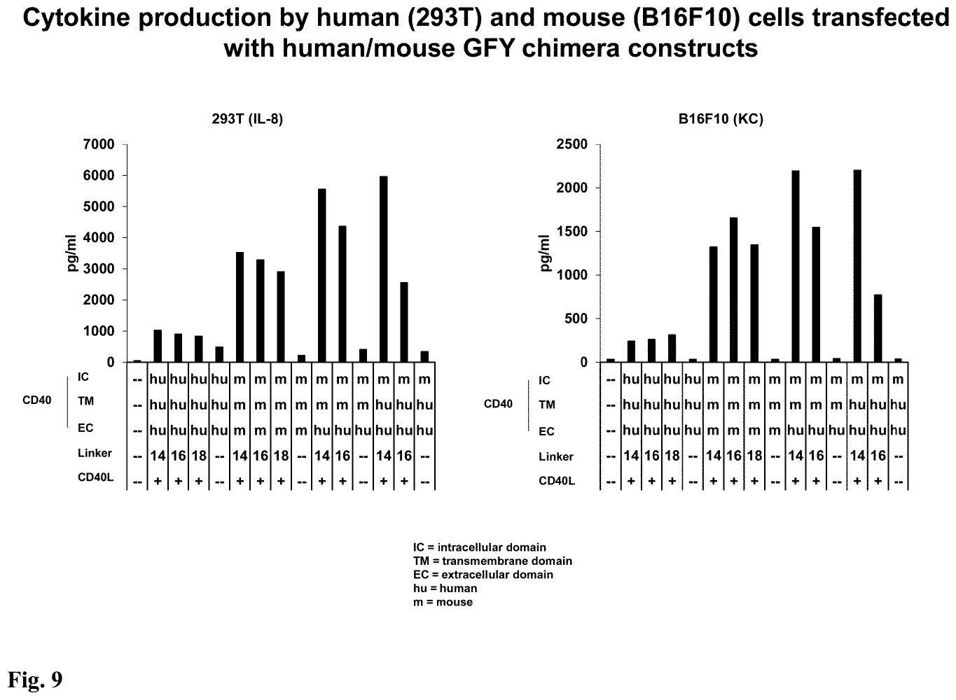

Functionality of the chimeric constructs was tested against 293T transfected with CD40 which were subsequently stimulated with sCD40L. Results are shown in FIG. 8. Notably, the chimeric constructs induced more IL-8 secretion than did the soluble CD40 ligand. Finally, chimeric constructs were prepared using mouse and human sequence elements for the CD40 domain of the fusion protein. Therefore, at least some of the fusion proteins were also chimeric with respect to origin of the intracellular (IC), transmembrane™, or extracellular (EC) domain. Remarkably, FIG. 9 shows that chimeric constructs in human cells using human EC elicited significantly higher IL-8 secretion, even where murine IC and TM segments were used. Similarly, the human EC was also superior in murine cells.

In preferred embodiments, CD40/CD40L protein constructs are illustrated in the accompanying sequence listing. SEQ ID.NO:1 is one illustrative example of a human CD40/CD40L construct having a 12mer linker. SEQ ID.NO:2 is one illustrative example of a human CD40/CD40L construct having a 14mer linker. SEQ ID.NO:3 is one illustrative example of a human CD40/CD40L construct having a 16mer linker. SEQ ID.NO:4 is one illustrative example of a human CD40/CD40L construct having a 18mer linker. SEQ ID.NO:5 is one illustrative example of a human CD40/CD40L construct having a 20mer linker. SEQ ID.NO:6 is one illustrative example of a mouse CD40/CD40L construct having a 12mer linker. SEQ ID.NO:7 is one illustrative example of a mouse CD40/CD40L construct having a 14mer linker. SEQ ID.NO:8 is one illustrative example of a mouse CD40/CD40L construct having a 16mer linker. SEQ ID.NO:9 is one illustrative example of a mouse CD40/CD40L construct having a 18mer linker. SEQ ID.NO10 is one illustrative example of a mouse CD40/CD40L construct having a 20 mer linker. Further constructs for 4-1BBL/4-1BB and Ox40L/Ox40 may be based on the publicly available Uniprot sequences in a manner substantially as described above for CD40L/CD40.

In some preferred embodiments, a genetically engineered activated dendritic cell may be made by infecting a tumor cell with a recombinant nucleic acid having first and second nucleic acid segments; wherein the first nucleic acid segment encodes a chimeric protein having an extracellular portion of CD40 coupled by a flexible linker to CD40L; and wherein the second nucleic acid segment encodes a tumor-associated antigen. The genetically engineered activated dendritic cell may further comprise a recombinant nucleic acid that encodes an antibody secreting moiety to affect the tumor microenvironment. The antibody secreting moiety may comprise one or more of: PD1, CTLA4 and TGFbtrap and IL 8.

In some embodiments, the genetically engineered activated DC may be useful for the treatment of tumor. The method involves administering to the patient a composition comprising the genetically engineered activated DCs as discussed above. Administration of the genetically engineered activated DC may be done locally (bladder cancer, brain cancer), or topically (skin tumors), or interventional injection into the tissue (liver cancer, breast cancer, pancreatic cancer), or inhaled (lung cancer, or brain cancer) or intrathecally. In some embodiments the tumor killing property of the engineered cell may be further enhanced via CD46 to target both the CARs and CD46. Furthermore, the methods and the engineered cells disclosed herein may be used as disclosed by Do et al (2018) Int. J. Mol. Sci. 19:2694, and Zhai et al (2102) Gene Ther. 19(11):1065-74.

Neoepitope-based immune therapy can be improved by use of an adjuvant that is either co-expressed or co-presented with the immunogenic peptides (which are preferably patient and tumor specific neoepitopes). Most typically, the expressed patient- and tumor specific neoepitopes are targeted towards processing and/or specific cell surface presentation or even secretion. Where desirable, neoepitope-based therapy can still further be augmented using checkpoint inhibition, immune stimulation via cytokines, and/or inhibitors of myeloid derived suppressor cells (MDCS), T-regulatory cells (Tregs), or M2 macrophages.

By way of non-limiting example, such therapeutic entities will be expressed in vivo from a recombinant nucleic acid, and especially suitable recombinant nucleic acid include plasmids and viral nucleic acids. Where a viral nucleic acid is employed, it is particularly preferred that the nucleic acid be delivered via viral infection of patient cells.

The compositions and methods presented herein will deliver an adjuvant in the context of expression and/or presence of one or more neoepitopes. Indeed, such treatment can advantageously be tailored to achieve one or more specific immune reactions, including a CD4⁺ biased immune response, a CD8⁺ biased immune response, antibody biased immune response, and/or a stimulated immune response (e.g., reducing checkpoint inhibition and/or by activation of immune competent cells using cytokines), all of which can benefit from the presence of the adjuvant. Where the adjuvant is not expressed (e.g., adjuvant is ATP or an ATP analog), the adjuvant is preferably injected into the tumor such that the vaccine composition and the adjuvant are present at the same time (e.g., vaccine composition and adjuvant present at measurable quantities at the same time).

All known adjuvants are suitable for use herein. Suitable exemplary adjuvants include various inorganic compounds such as alum, aluminum hydroxide, aluminum phosphate, calcium phosphate hydroxide, mineral oils, and especially paraffin oil. Further suitable adjuvants include small molecule compounds such as squalene, as well as various bacterial products such as killed bacteria Bordetella pertussis, Mycobacterium bovis toxoids, etc. Adjuvants may also formed from one or more emulsified neoantigens to produce complex compositions such as Freund's complete adjuvant, or Freund's incomplete adjuvant.

Especially suitable adjuvants include various DAMPs (damage-associated molecular pattern proteins). DAMPs are known to trigger inflammation, innate and adaptive immune responses, and tissue healing after damage. Particularly preferred DAMPs include calreticulin or portions with adjuvant activity thereof, and HMGB1 or portions with adjuvant activity thereof. Still further DAMPs include S100 proteins and various cytokines, and especially IL-1, IL-2, and IL-12.

HMGB1 is a damage-associated molecular pattern (DAMP) protein that is normally inside a cell, but released after cell death to allow immune distinction between dangerous and harmless antigens. Cells undergoing severe stress secrete HMGB1. Extracellular HMGB1 triggers inflammation and adaptive immunological responses. HMGB1 was also reported to enhance the immunogenicity of mutated proteins in the tumor (neoantigens or neoepitopes), promoting anti-tumor responses and immunological memory (see e.g., Immunol Rev. (2017) 280(1):74-82). For example, HMGB1 was reported to induce dendritic cells maturation and T helper-1-cell responses.

Specific fragments of HMGB1 were reported to activate dendritic cells (see, U.S. Pat. No. 9,539,321). Peptides comprising a sequence of SAFFLFCSE were immunostimulatory in vitro and that such sequences could be attached to nano- or microparticles. HMGB1 also promotes maturation of antigen presenting cells (US 2004/0242481). Portions of HMGB1 were employed in a fusion protein to activate T-cells as described in US 2011/0236406.

Cancer therapy can induce stress response in the ER, translocating calreticulin to the outer leaflet of the plasma membrane before the morphological appearance of apoptosis. Such surface-exposed calreticulin serves as a powerful mobilizing signal to the immune system. Therefore, externally added calreticulin in the context of photodynamic therapy of tumors is an immune enhancer (Front Immunol (2015) 5(15):1-7).

HMGB1 and calreticulin adjuvants require either formulating a mixture of the vaccine compound and the adjuvant in a more traditional manner, or systemic administration of adjuvant outside the context of the antigens for calreticulin. However, such approaches are generally unsuitable for cancer therapy, especially where the cancer antigens are recombinant antigens.

Advantageously, as described herein, polypeptide or protein adjuvants can be administered in the immediate context of neoepitopes (or TAAs or tumor specific antigens) by co-expression of the polypeptide or protein adjuvants together with the neoepitopes. Such co-expression may be performed in live cells, and especially in APCs of a patient diagnosed with a tumor, or in yeast or bacterial vaccine compositions administered to the patient.

For example, as is described in more detail below, a recombinant nucleic acid may be constructed that includes one or more expression cassettes for expression of neoepitopes, preferably in a manner that directs the neoepitopes towards MHC-I and/or MHC-II presentation and that further includes an expression cassette that encodes one or more polypeptide or protein adjuvants. The polypeptide or protein adjuvant can be expressed as a membrane-bound protein or as a soluble secreted protein. The recombinant nucleic acid may further include a sequence encoding at least one of a co-stimulatory molecule, an immune stimulatory cytokine, and a protein that interferes with or down-regulates checkpoint inhibition. Suitable co-stimulatory molecules include OX40L, 4-1BBL, CD80, CD86, CD30, CD40, CD30L, CD40L, ICOS-L, B7-H3, B7-H4, CD70, GITR-L, TIM-3, TIM-4, CD48, CD58, TL1A, ICAM-1, and LFA3, and suitable immune stimulatory cytokines include IL-2, IL-12, IL-15, IL-15 super agonist (ALT803), IL-21, IPS1, and LMP1. Preferred proteins that interfere with checkpoint inhibition include antibodies or antagonists of CTLA-4, PD-1, TIM1 receptor, 2B4, or CD160.

Additionally or alternatively, non-protein stress signals may be delivered to the tumor as part of an immunotherapy, via systemic or intratumoral administration. For example, non-protein adjuvants include various purine metabolites, particularly ATP and ATP analogs (e.g., non-hydrolysable α, β-methylene-ATP (αβ-ATP)). Extracellular ATP serves as a danger signal to alert the immune system of tissue damage, and triggering DC activation.

Cancer immune therapy can uses a recombinant adenovirus. Such adenoviruses can carry cancer epitopes as payloads, as well as at least one polypeptide or protein adjuvant, and optionally additional functional elements as discussed below. The cancer epitopes are typically tumor and patient specific neoepitopes filtered according to one or more criteria as also described below.

Neoepitopes identification may start with a variety of biological materials, including fresh biopsies, frozen, or otherwise preserved tissue or cell samples, circulating tumor cells, exosomes, various body fluids (and especially blood), etc. Suitable omics analysis methods include nucleic acid sequencing, and particularly NGS methods operating on DNA (e.g., Illumina sequencing, ion torrent sequencing, 454 pyrosequencing, nanopore sequencing, etc.), RNA sequencing (e.g., RNAseq, reverse transcription based sequencing, etc.), and in some cases protein sequencing or mass spectroscopy based sequencing (e.g., SRM, MRM, CRM, etc.).

For nucleic acid based sequencing, high-throughput genome sequencing of a tumor tissue permits rapid identification of neoepitopes. However, where the sequence information is compared against a standard reference, normally occurring inter-patient variation (e.g., due to SNPs, short indels, different number of repeats, etc.) as well as heterozygosity will result in a relatively large number of potential false positive neoepitopes (i.e., neoepitopes that are also found on health tissue in the same patient). Notably, such inaccuracies can be eliminated where a tumor sample of a patient is compared against a matched normal (i.e., non-tumor) sample of the same patient.

DNA analysis may be performed by whole genome sequencing and/or exome sequencing (typically at a coverage depth of at least 10×, more typically at least 20×) of both tumor and matched normal sample. Alternatively, DNA data may also be provided from an already established sequence record (e.g., SAM, BAM, FASTA, FASTQ, or VCF file) from a prior sequence determination of the same patient. Suitable data sets include unprocessed or processed data sets, and exemplary preferred data sets include those having BAM format, SAM format, GAR format, FASTQ format, or FASTA format, as well as BAMBAM, SAMBAM, and VCF data sets. However, BAM format or BAMBAM diff objects are especially suitable, as described in US 2012/0059670 and US 2012/0066001. The data sets reflect a tumor and a matched normal sample of the same patient. Thus, genetic germ line alterations not giving rise to the tumor (e.g., silent mutation, SNP, etc.) can be excluded. The tumor sample may be from an initial tumor, from the tumor upon start of treatment, from a recurrent tumor and/or metastatic site, etc. In most cases, the matched normal sample of the patient is blood, or a non-diseased tissue from the same tissue type as the tumor.

Likewise, sequence data may be analyzed in numerous manners. In most preferred methods, however, analysis is performed in silico by location-guided synchronous alignment of tumor and normal samples as in US 2012/0059670 and US 2012/0066001 using BAM files and BAM servers. Such analysis advantageously reduces false positive neoepitopes and significantly reduces demands on memory and computational resources.

Any language directed to a “computer” should be read to include any suitable combination of computing devices, including servers, interfaces, systems, databases, agents, peers, engines, controllers, or other types of computing devices operating individually or collectively. Computing devices comprise a processor configured to execute software instructions stored on a tangible, non-transitory computer readable storage medium (e.g., hard drive, solid state drive, RAM, flash, ROM, etc.). The software instructions preferably configure the computing device to provide the roles, responsibilities, or other functionality as discussed below with respect to the disclosed apparatus. Further, the disclosed technologies can be embodied as a computer program product that includes a non-transitory computer readable medium storing the software instructions that causes a processor to execute the disclosed steps associated with implementations of computer-based algorithms, processes, methods, or other instructions. In especially preferred embodiments, the various servers, systems, databases, or interfaces exchange data using standardized protocols or algorithms, possibly based on HTTP, HTTPS, AES, public-private key exchanges, web service APIs, known financial transaction protocols, or other electronic information exchanging methods. Data exchanges among devices can be conducted over a packet-switched network, the Internet, LAN, WAN, VPN, or other type of packet switched network; a circuit switched network; cell switched network; or other type of network.

Patient- and cancer-specific in silico collection of sequences can be established that encode neoepitopes having a predetermined length of, for example, between 5 and 25 amino acids and include at least one changed amino acid. Such collection will typically include for each changed amino acid at least two, at least three, at least four, at least five, or at least six members in which the position of the changed amino acid is not identical. Such collection advantageously increases potential candidate molecules suitable for immune therapy and can then be used for further filtering (e.g., by sub-cellular location, transcription/expression level, MHC-I and/or II affinity, etc.) as is described in more detail below.

For example, and using synchronous location guided analysis to tumor and matched normal sequence data, various cancer neoepitopes have been identified from a variety of cancers and patients, including the following cancer types: BLCA, BRCA, CESC, COAD, DLBC, GBM, HNSC, KICH, KIRC, KIRP, LAML, LGG, LIHC, LUAD, LUSC, OV, PRAD, READ, SARC, SKCM, STAD, THCA, and UCEC. Exemplary neoepitope data for these cancers can be found in International application PCT/US16/29244, incorporated by reference herein.

Depending on the type and stage of the cancer, as well as the patient's immune status, not all of the identified neoepitopes will necessarily lead to a therapeutically equally effective reaction in a patient. Indeed, only a fraction of neoepitopes will generate an immune response. To increase likelihood of a therapeutically desirable response, the initially identified neoepitopes can be further filtered. Downstream analysis need not take into account silent mutations for the purpose of the methods presented herein. However, preferred mutation analyses will provide in addition to the particular type of mutation (e.g., deletion, insertion, transversion, transition, translocation) also information of the impact of the mutation (e.g., non-sense, missense, etc.) and may as such serve as a first content filter through which silent mutations are eliminated. For example, neoepitopes can be selected for further consideration where the mutation is a frame-shift, non-sense, and/or missense mutation.

In a further filtering approach, neoepitopes may also be subject to detailed analysis for sub-cellular location parameters. For example, neoepitope sequences may be selected for further consideration if the neoepitopes are identified as having a membrane associated location (e.g., are located at the outside of a cell membrane of a cell) and/or if an in silico structural calculation confirms that the neoepitope is likely to be solvent exposed, or presents a structurally stable epitope (e.g., J Exp Med 2014), etc.

Neoepitopes are especially suitable for use herein where omics or other analyses reveal that the neoepitope is actually expressed. Identification of expression and expression level of a neoepitope can be performed in all manners known in the art. Preferred methods include quantitative RNA (hnRNA or mRNA) analysis and/or quantitative proteomics analysis. Most typically, the threshold level for inclusion of neoepitopes will be an expression level of at least 20%, at least 30%, at least 40%, or at least 50% of expression level of the corresponding matched normal sequence, thus ensuring that the (neo)epitope is at least potentially ‘visible’ to the immune system. Consequently, it is generally preferred that the omics analysis also includes an analysis of gene expression (transcriptomic analysis) to help identify the level of expression for the gene with a mutation.

Numerous methods of transcriptomic analysis are known in the art, and all known methods are suitable for use herein. For example, mRNA and primary transcripts (hnRNA), and RNA sequence information may be obtained from reverse transcribed polyA⁺-RNA, which is in turn obtained from a tumor sample and a matched normal (healthy) sample of the same patient. Likewise, while polyA⁺-RNA is typically preferred as a representation of the transcriptome, other forms of RNA (hn-RNA, non-polyadenylated RNA, siRNA, miRNA, etc.) are also suitable. Preferred methods include quantitative RNA (hnRNA or mRNA) analysis and/or quantitative proteomics analysis, especially including RNAseq. In other aspects, RNA quantification and sequencing is performed using RNAseq, qPCR and/or rtPCR based methods, although various alternative methods (e.g., solid phase hybridization-based methods) are also suitable. Transcriptomic analysis may be suitable (alone or in combination with genomic analysis) to identify and quantify genes having a cancer- and patient-specific mutation.

Similarly, proteomics analysis can be performed in numerous manners to ascertain actual translation of the RNA of the neoepitope, and all known proteomics analyses are suitable. Preferred proteomics methods include antibody-based methods and mass spectroscopic methods. Proteomics analysis may not only provide qualitative or quantitative information about the protein per se, but may also include protein activity data where the protein has catalytic or other functional activity. See, e.g., U.S. Pat. No. 7,473,532, incorporated by reference herein. Further suitable methods of identification and even quantification of protein expression include various mass spectroscopic analyses (e.g., selective reaction monitoring (SRM), multiple reaction monitoring (MRM), and consecutive reaction monitoring (CRM)). The above methods will provide patient and tumor specific neoepitopes, which may be further filtered by sub-cellular location of the protein containing the neoepitope (e.g., membrane location), the expression strength (e.g., overexpressed as compared to matched normal of the same patient), etc.

Neoepitopes may be compared against a database that contains known human sequences (e.g., of the patient or a collection of patients) to so avoid use of a human-identical sequence. Moreover, filtering may also include removal of neoepitope sequences that are due to SNPs in the patient where the SNPs are present in both the tumor and the matched normal sequence. For example, dbSNP (The Single Nucleotide Polymorphism Database) is a free public archive for genetic variation within and across different species developed and hosted by the National Center for Biotechnology Information (NCBI) in collaboration with the National Human Genome Research Institute (NHGRI). Although the name of the database implies a collection of one class of polymorphisms only (single nucleotide polymorphisms (SNPs)), it in fact contains a relatively wide range of molecular variation: (1) SNPs, (2) short deletion and insertion polymorphisms (indels/DIPs), (3) microsatellite markers or short tandem repeats (STRs), (4) multinucleotide polymorphisms (MNPs), (5) heterozygous sequences, and (6) named variants. The dbSNP accepts apparently neutral polymorphisms, polymorphisms corresponding to known phenotypes, and regions of no variation. Using such database and other filtering options as described above, the patient and tumor specific neoepitopes may be filtered to remove those known sequences, yielding a sequence set with a plurality of neoepitope sequences having substantially reduced false positives.

Once the neoepitope is adequately filtered (e.g., by tumor versus normal, and/or expression level, and/or sub-cellular location, and/or patient specific HLA-match, and/or known variants), a further filtering step may take into account the gene type that is affected by the neoepitope. For example, suitable gene types include cancer driver genes, genes associated with regulation of cell division, genes associated with apoptosis, and genes associated with signal transduction. However, in especially preferred aspects, cancer driver genes are particularly preferred (which may span by function a variety of gene types, including receptor genes, signal transduction genes, transcription regulator genes, etc.). Suitable gene types may also be known passenger genes and genes involved in metabolism.

Various methods and prediction algorithms are known in the art to determine whether a gene be a cancer driver. For example, suitable algorithms include MutsigCV (Nature 2014, 505(7484):495-501), ActiveDriver (Mol Syst Biol 2013, 9:637), MuSiC (Genome Res 2012, 22(8): 1589-1598), OncodriveClust (Bioinformatics 2013, 29(18):2238-2244), OncodriveFM (Nucleic Acids Res 2012,40(21):e169), OncodriveFML (Genome Biol 2016, 17(1):128), Tumor Suppressor and Oncogenes (TUSON) (Cell 2013, 155(4):948-962), 20/20+(https://github.com/KarchinLab/2020p1us), and oncodriveROLE (Bioinformatics (2014) 30 (17): i549-i555). Alternatively or additionally, identification of cancer driver genes may also employ various sources for known cancer driver genes and their association with specific cancers. For example, the Intogen Catalog of driver mutations (2016.5; URL: www.intogen.org) contains the results of the driver analysis performed by the Cancer Genome Interpreter across 6,792 exomes of a pan-cancer cohort of 28 tumor types.

Nevertheless, despite filtering, not all neoepitopes will be visible to the immune system as neoepitopes also need to be processed where present in a larger context (e.g., within a polytope) and presented on the MHC complex of the patient. Only a fraction of all neoepitopes will have sufficient affinity for presentation. Consequently, neoepitopes will be more likely effective where the neoepitopes are properly processed, bound to, and presented by the MHC complexes. Treatment success will be increased with an increasing number of neoepitopes that can be presented via the MHC complex, wherein such neoepitopes have a minimum affinity to the patient's HLA-type. Effective binding and presentation is a combined function of the sequence of the neoepitope and the particular HLA-type of a patient. Therefore, HLA-type determination of the patient tissue is typically required. Most typically, the HLA-type determination includes at least three MHC-I sub-types (e.g., HLA-A, HLA-B, HLA-C) and at least three MHC-II sub-types (e.g., HLA-DP, HLA-DQ, HLA-DR), preferably with each subtype being determined to at least 2-digit, at least 4-digit, at least 6 digit, or at least 8 digit depth.

Once the HLA-type of the patient is ascertained, a structural solution for the HLA-type is calculated and/or obtained from a database, which is then used in a docking model in silico to determine binding affinity of the (typically filtered) neoepitope to the HLA structural solution. Suitable systems for determination of binding affinities include the NetMHC platform (see e.g., Nucleic Acids Res. 2008 Jul. 1; 36(Web Server issue): W509-W512.). Neoepitopes with high affinity (e.g., less than 100 nM, less than 75 nM, less than 50 nM) for a previously determined HLA-type are then selected for therapy creation, along with the knowledge of the patient's MHC-I/II subtype.

HLA determination can be performed using various methods in wet-chemistry known in the art. All of these methods are suitable for use herein. The HLA-type can be predicted from omics data in silico using a reference sequence containing most or all of the known and/or common HLA-types. For example, a database can provide a relatively large number of patient sequence reads mapping to chromosome 6p21.3 (or any other location near HLA alleles). Most typically the sequence reads will have about 100-300 bases and comprise metadata, including read quality, alignment information, orientation, location, etc. For example, suitable formats include SAM, BAM, FASTA, GAR, etc. By way of non-limiting example, the patient sequence reads may provide a depth of coverage of at least 5×, more typically at least 10×, even more typically at least 20×, and most typically at least 30×.

In addition to the patient sequence reads, the present methods further employ one or more reference sequences that include a plurality of sequences of known and distinct HLA alleles. For example, a typical reference sequence may be a synthetic (without corresponding human or other mammalian counterpart) sequence that includes sequence segments of at least one HLA-type with multiple HLA-alleles of that HLA-type. Suitable reference sequences include without limitation a collection of known genomic sequences for at least 50 different alleles of HLA-A. Alternatively or additionally, the reference sequence may also include a collection of known RNA sequences for at least 50 different alleles of HLA-A. The reference sequence is not limited to 50 alleles of HLA-A, but may have alternative composition with respect to HLA-type and number/composition of alleles. Most typically, the reference sequence will be in a computer readable format and will be provided from a database or other data storage device. For example, suitable reference sequence formats include FASTA, FASTQ, EMBL, GCG, or GenBank format, and may be directly obtained or built from data of a public data repository (e.g., IMGT, the International ImMunoGeneTics information system, or The Allele Frequency Net Database, EUROSTAM). Alternatively, the reference sequence may also be built from individual known HLA-alleles based on one or more predetermined criteria such as allele frequency, ethnic allele distribution, common or rare allele types, etc.

Using the reference sequence, the patient sequence reads can be threaded through a de Bruijn graph to identify the alleles with the best fit. Each individual carries two alleles for each HLA-type, and that these alleles may be very similar, or in some cases even identical. Such high degree of similarity poses a significant problem for traditional alignment schemes. HLA alleles, and even very closely related alleles can be resolved using an approach in which the de Bruijn graph is constructed by decomposing a sequence read into relatively small k-mers (typically having a length of between 10-20 bases), and by implementing a weighted vote process in which each patient sequence read provides a vote (“quantitative read support”) for each of the alleles on the basis of k-mers of that sequence read that match the sequence of the allele. The cumulatively highest vote for an allele then indicates the most likely predicted HLA allele. In addition, it is generally preferred that each fragment that is a match to the allele is also used to calculate the overall coverage and depth of coverage for that allele.

Scoring may further be improved or refined as needed, especially where many of the top hits are similar (e.g., where a significant portion of their score comes from a highly shared set of k-mers). For example, score refinement may include a weighting scheme in which alleles that are substantially similar (e.g., >99%, or other predetermined value) to the current top hit are removed from future consideration. Counts for k-mers used by the current top hit are then re-weighted by a factor (e.g., 0.5), and the scores for each HLA allele are recalculated by summing these weighted counts. This selection process is repeated to find a new top hit. The accuracy of the method can be even further improved using RNA sequence data that allows identification of the alleles expressed by a tumor, which may sometimes be just 1 of the 2 alleles present in the DNA. DNA or RNA, or a combination of both DNA and RNA can be processed to make HLA predictions that are highly accurate and can be derived from tumor or blood DNA or RNA. Further aspects, suitable methods and considerations for high-accuracy in silico HLA typing are described in WO 2017/035392, incorporated by reference herein.

Once patient and tumor specific neoepitopes and HLA-type are identified, further computational analysis can be performed by in silico docking neoepitopes to the HLA and determining best binders (e.g., lowest K_{D}, for example, less than 500 nM, or less than 250 nM, or less than 150 nM, or less than 50 nM), for example, using NetMHC. Such approaches will not only identify specific neoepitopes that are genuine to the patient and tumor, but also those neoepitopes that are most likely to be presented on a cell and as such most likely to elicit an immune response with therapeutic effect. These HLA-matched neoepitopes can be biochemically validated in vitro prior to inclusion of the nucleic acid encoding the epitope as payload into the virus as is further discussed below.

Upon identification of desired neoepitopes, one or more immune therapeutic agents may be prepared using the sequence information of the neoepitope. Among other agents, the patient may be treated with a virus that is genetically modified with a nucleic acid construct as further discussed below that leads to expression of at least one of the identified neoepitopes to initiate an immune response against the tumor. For example, suitable viruses include adenoviruses, adeno-associated viruses, alphaviruses, herpes viruses, lentiviruses, etc. However, adenoviruses are particularly preferred. Moreover, it is further preferred that the virus be a replication deficient and non-immunogenic virus, which is typically accomplished by targeted deletion of selected viral proteins (e.g., E1, E3 proteins). Such desirable properties may be further enhanced by deleting E2b adenoviral gene function. High titers of recombinant viruses can be achieved using genetically modified human 293 cells (see, e.g., J Virol. (1998) 72(2):926-33).

The virus may be used to infect patient (or non-patient) cells ex vivo or in vivo. For example, the virus may be injected subcutaneously or intravenously, or may be administered intranasaly or via inhalation to so infect the patients cells, especially APCs. Alternatively, immune competent cells (e.g., NK cells, T cells, macrophages, DCs, etc.) may be infected in vitro and then transfused to the patient. Alternatively, immune therapy need not rely on a virus but may be effected with nucleic acid transfection or vaccination using RNA or DNA, or other recombinant vectors that lead to neoepitope expression (e.g., as single peptides, tandem mini-gene, etc.) in desired cells, especially immune competent cells.

Most typically, nucleic acid sequences (for expression from virus infected cells) are under the control of appropriate regulatory elements well known in the art. For example, suitable promoter elements include constitutive strong promoters (e.g., SV40, CMV, UBC, EF1A, PGK, CAGG promoter), but inducible promoters are also suitable for use herein, particularly where induction conditions are typical for a tumor microenvironment. For example, inducible promoters include those sensitive to hypoxia and promoters that are sensitive to TGF-β or IL-8 (e.g., via TRAF, JNK, Erk, or other responsive elements promoter). In other examples, suitable inducible promoters include the tetracycline-inducible promoter, the myxovirus resistance 1 (M×1) promoter, etc.

The manner of neoepitope arrangement and rational-designed trafficking of the neoepitopes can impact immune therapeutic composition efficacy. For example, single neoepitopes can be expressed individually from the recombinant constructs that are delivered as a single plasmid, viral expression construct, etc. Alternatively, multiple neoepitopes can be separately expressed from individual promoters to form individual mRNAs that are then individually translated into the respective neoepitopes. A single mRNA comprising individual translation starting points for each neoepitope sequence (e.g., using 2A or IRES signals) can also be used.

Where multiple neoepitopes were expressed from a single transcript to form a single transcript that is then translated into a single polytope, expression, processing, and antigen presentation was found to be effective. Polytope expression requires processing by the appropriate proteases (e.g., proteasome, endosomal proteases, lysosomal proteases) within a cell to yield the neoepitope sequences, and polytopes led to improved antigen processing and presentation for most neoepitopes as compared to expression of individual neoepitopes, particularly where the individual neoepitopes had a relatively short length (e.g., less than 25 amino acids; results not shown). Moreover, such approach also allows rational design of protease sensitive sequence motifs between the neoepitope peptide sequences to assure or avoid processing by specific proteases as the proteasome, endosomal proteases, and lysosomal proteases have distinct cleavage preferences. Therefore, polytopes may be designed that include not only linker sequences to spatially separate neoepitopes, but also sequence portions (e.g., 3-15 amino acids) that will be preferentially cleaved by a specific protease.

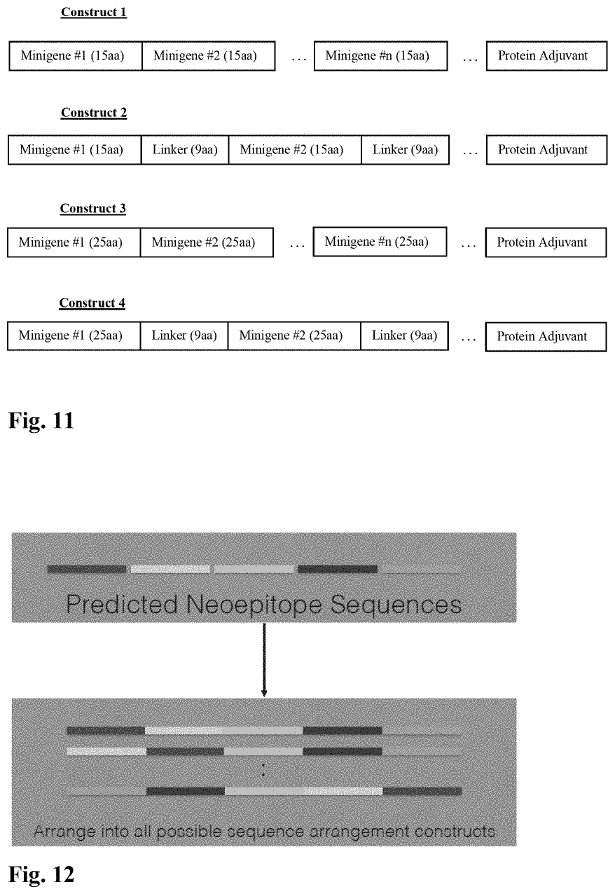

Recombinant nucleic acids and expression vectors (e.g., viral expression vectors) can be used that comprise a nucleic acid segment encoding a polytope operably coupled to a desired promoter element, and wherein individual neoepitopes are optionally separated by a linker and/or protease cleavage or recognition sequence. For example, FIG. 11 illustrates various contemplated arrangements for neoepitopes for expression from an adenoviral expression system (here: AdV5, with deletion of E1 and E2b genes). Here, Construct 1 illustrates an exemplary neoepitope arrangement that comprises multiple neoepitopes (‘minigene’) with a total length of 15 amino acids in concatemeric series without intervening linker sequences, while Construct 2 shows the arrangement of Construct 1 but with inclusion of nine amino acid linkers between each neoepitope sequence. Of course, and as already noted above, the exact neoepitope length is not limited to 15 amino acids, but rather may vary considerably. However, in most cases, where neoepitopes of between 8-12 amino acids (e.g., for MHC-I presentation) are flanked by additional amino acids, the total length will typically not exceed 25 amino acids, or 30 amino acids, or 50 amino acids. While FIG. 11 denotes G-S linkers, various other linker sequences are also suitable for use herein. Such relatively short neoepitopes are especially beneficial where neoepitope is to be presented via MHC-I.

Suitable linker sequences will provide steric flexibility and separation of two adjacent neoepitopes. However, one must not choose amino acids for the linker that could be immunogenic or that could form an epitope that is already present in a patient. The polytope construct can be filtered once more for the presence of epitopes that could be found in a patient (e.g., as part of normal sequence or due to SNP or other sequence variation). Such filtering will apply the same technology and criteria as already discussed above.

Construct 3 illustrates an exemplary neoepitope arrangement including multiple neoepitopes in concatemeric series without intervening linker sequences, and Construct 4 shows the arrangement of Construct 3 with inclusion of nine amino acid linkers between each neoepitope sequence. As noted above, the exact neoepitope length is not limited to 25 amino acids, but may vary considerably. However, in most cases, where neoepitope sequences of between 14-20 amino acids (e.g., for MHC-II presentation) are flanked by additional amino acids, the total length will typically not exceed 30 amino acids, or 45 amino acids, or 60 amino acids. While FIG. 11 denotes G-S linkers, various other linker sequences are also suitable for use herein. Such relatively short neoepitopes are especially beneficial where neoepitope is to be presented via MHC-I.

In this example, the 15-aa minigenes are MHC Class I targeted tumor mutations selected with 7 amino acids of native sequence on either side, and the 25-aa minigenes are MHC Class II targeted tumor mutations selected with 12 amino acids of native sequence on either side. The exemplary 9 amino acid linkers have sufficient length to avoid formation of “unnatural” MHC Class I epitopes between adjacent minigenes. Polytope sequences process and present more efficiently than single neoepitopes (data not shown). Addition of amino acids beyond 12 amino acids for MHC-I presentation and of amino acids beyond 20 amino acids for MHC-I presentation improve protease processing.

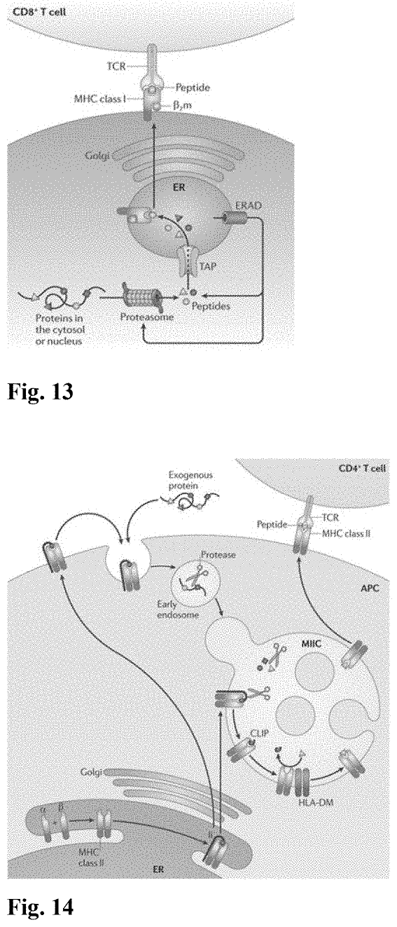

To maximize intracellular retention of customized protein sequences for processing and HLA presentation, neoepitope sequences may be arranged to minimize hydrophobic sequences that may direct trafficking to the cell membrane or extracellular space. Most preferably, hydrophobic sequence or signal peptide detection is done either by comparison of sequences to a weight matrix (see e.g., Nucleic Acids Res. (1986) 14(11):4683-90) or by using neural networks trained on peptides that contain signal sequences (see e.g., J. Mol. Biol. (2004) 338(5):1027-36). FIG. 12 depicts an exemplary arrangement in which a plurality of polytopes are analyzed. Here, all neoepitope positional permutations are calculated to produce a collection of arrangements. This collection is then processed through a weight matrix and/or neural network prediction to generate a score representing the likelihood of presence and/or strength of hydrophobic sequences or signal peptides. All positional permutations are then ranked by score, and the permutation(s) with a score below a predetermined threshold or lowest score for likelihood of presence and/or strength of hydrophobic sequences or signal peptides is/are used to construct a customized neoepitope expression cassette.

It is generally preferred that the polytope comprise at least two, or at least three, or at least five, or at least eight, or at least ten neoepitope sequences. Indeed, the payload capacity of the recombinant DNA is generally contemplated the limiting factor, along with the availability of filtered and appropriate neoepitopes. Therefore, adenoviral expression vectors, and particularly Adv5 are especially preferred as such vectors can accommodate up to 14 kb in recombinant payload.

Neoepitopes/polytopes can be directed towards a specific sub-cellular compartment (e.g., cytosol, endosome, lysosome), and with that, towards a particular MHC presentation type. Such directed expression, processing, and presentation is particularly advantageous as compositions may be prepared that direct an immune response towards a CD8⁺ type response (where the polytope is directed to the cytoplasmic space) or towards a CD4⁺ type response (where the polytope is directed to the endosomal/lysosomal compartment). Polytopes that would ordinarily be presented via the MHC-I pathway can be presented via the MHC-II pathway (and thereby mimic cross-presentation of neoepitopes). Neoepitope and polytope sequences may be designed and directed to one or both MHC presentation pathways using suitable sequence elements. MHC-I presented peptides will typically arise from the cytoplasm via proteasome processing and delivery through the endoplasmic reticulum. Thus, expression of the epitopes intended for MHC-I presentation will generally be directed to the cytoplasm as is further discussed in more detail below. On the other hand, MHC-II presented peptides will typically arise from the endosomal and lysosomal compartment via degradation and processing by acidic proteases (e.g., legumain, cathepsin L and cathepsin S) prior to delivery to the cell membrane.

Proteolytic degradation of the polytope can also be enhanced using various methods, including addition of a cleavable or non-cleavable ubiquitin moiety to the N-terminus, and/or placement of one or more destabilizing amino acids (e.g., N, K, C, F, E, R, Q) at the polytope's N-terminus where the presentation is directed toward MHC-I. Where presentation is directed toward MHC-II, endosomal or lysosomal protease cleavage sites can be engineered into the polytope.

Signal and/or leader peptides can traffic neoepitopes and/or polytopes to the endosomal and lysosomal compartments, or retain the neoepitopes/polytopes in the cytoplasmic space. For example, to export a polytope to an endosome or lysosome, a leader peptide such as the CD1b leader peptide can sequester the polytope from the cytoplasm. Additionally or alternatively, targeting presequences and/or targeting peptides can be added to the N-terminus and/or C-terminus. Targeting presequences typically comprise between 6 and 136 basic and hydrophobic amino acids. The sequence for peroxisomal targeting can be at the C-terminus. Other signals (e.g., signal patches) include sequence elements that are separate in the peptide sequence and become functional upon proper peptide folding. Protein modifications like glycosylations can induce targeting. Suitable targeting signals include but are not limited to peroxisome targeting signal 1 (PTS1) and peroxisome targeting signal 2 (PTS2).

In addition, proteins can be sorted to endosomes and lysosomes by signals within the cytosolic domains of the proteins, typically short, linear sequences. “Tyrosine-based” sorting signals conform to the NPXY or YXX∅ consensus motifs. “Dileucine-based” signals fit [DE]XXXL[LI] or DXXLL consensus motifs. All of these signals are recognized by protein coat components on the cytosolic face of membranes. The adaptor protein (AP) complexes AP-1, AP-2, AP-3, and AP-4 recognize YXX∅ and [DE]XXXL[LI] signals with characteristic fine specificity, whereas the GGA adaptor family recognizes DXXLL signals. “FYVE” domain is associated with vacuolar protein sorting and endosome function. Human CD1 tail sequences (see e.g., Immunology, 122:522-31) can also target endosomes. LAMP1-TM (transmembrane) domains target lysosomes. CD1a tail sequences target recycling endosomes. Cd1c tail sequence target sorting endosomes.

The polytope may be a chimeric polytope that includes at least a portion of—and more typically an entire—TAA (e.g., CEA, PSMA, PSA, MUC1, AFP, MAGE, HER2, HCC1, p62, p90, etc.). TAAs are generally processed and presented via MHC-II. Therefore, instead of using compartment specific signal and/or leader sequences, the processing mechanism for TAAs can use MHC-II targeting.

Trafficking to or retention in the cytosolic compartment may not necessarily require one or more specific sequence elements. However, N- or C-terminal cytoplasmic retention signals (e.g., SNAP-25, syntaxin, synaptoprevin, synaptotagmin, vesicle associated membrane proteins (VAMPs), synaptic vesicle glycoproteins (SV2), high affinity choline transporters, Neurexins, voltage-gated calcium channels, acetylcholinesterase, and NOTCH) may be added, including a membrane-anchored protein or a membrane anchor domain of a membrane-anchored protein.

The polytope may also comprise one or more transmembrane segments to direct the neoepitope to the cell exterior after processing to be visible to immune competent cells. Numerous transmembrane domains are known in the art, all suitable for use herein, including those having a single alpha helix, multiple alpha helices, alpha/beta barrels, etc. For example, contemplated transmembrane domains include but are not limited to transmembrane region(s) of the alpha, beta, or zeta chain of the T-cell receptor, CD28, CD3 epsilon, CD45, CD4, CD5, CD8 (e.g., CD8 alpha, CD8 beta), CD9, CD16, CD22, CD33, CD37, CD64, CD80, CD86, CD134, CD137, CD154, KIRDS2, OX40, CD2, CD27, LFA-1 (CD11a, CD18), ICOS (CD278), 4-1BB (CD137), GITR, CD40, BAFFR, HVEM (LIGHTR), SLAMF7, NKp80 (KLRF1), CD160, CD19, IL2R beta, IL2R gamma, IL7R α, ITGA1, VLA1, CD49a, ITGA4, IA4, CD49D, ITGA6, VLA-6, CD49f, ITGAD, CD11d, ITGAE, CD103, ITGAL, CD11 a, LFA-1, ITGAM, CD11 b, ITGAX, CD11 c, ITGB 1, CD29, ITGB2, CD18, LFA-1, ITGB7, TNFR2, DNAM1 (CD226), SLAMF4 (CD244, 2B4), CD84, CEACAM1, CRTAM, Ly9 (CD229), CD160 (BY55), PSGL1, CD100 (SEMA4D), SLAMF6 (NTB-A, Ly108), SLAM (SLAMF1, CD150, IPO-3), BLAME (SLAMF8), SELPLG (CD162), LTBR, or PAG/Cbp. Where a fusion protein is desired, the recombinant chimeric gene can have a first portion encoding the transmembrane region(s) and a second portion—in frame with the first—encoding the inhibitory protein. This will not achieve MHC-complex presentation, and as such provides a neoepitope presentation independent of MHC/T-cell receptor interaction, which may open additional avenues for immune recognition to trigger antibody production against the neoepitopes.

Alternatively or additionally, the polytope may also include export signal sequences to force a transfected cell to produce and secrete one or more neoepitopes. For example, adding the SPARC leader sequence to a neoepitope or polytope sequence achieves in vivo neoepitope/polytope secretion into the extracellular space. Advantageously, such secreted neoepitopes or polytopes are then taken up by immune competent cells (especially APCs, e.g., DCs) that process and display the neoepitopes, typically via MHC-II pathways.

Alternatively or additionally, one may administer the neoepitope or polytope as peptide, optionally bound to a carrier protein. Among other suitable carrier proteins, human albumin or lactoferrin are preferred. Carrier proteins may be in native conformation, or pretreated to form nanoparticles with exposed hydrophobic domains (see e.g., (2015) Adv Protein Chem Struct Biol. 98:121-43) to which the neoepitope or polytope can be coupled. Most typically, the neoepitope or polytope is coupled to the carrier protein non-covalently. Carrier-bound neoepitopes or polytopes will be taken up by the immune competent cells, and especially APCs (e.g., DCs), that process and display the neoepitopes, typically via MHC-II pathways.

Immune therapeutic compositions can deliver one or more neoepitopes to various sub-cellular locations to generate distinct immune responses. For example, Prior Art FIG. 13 illustrates a polytope predominantly processed in the proteasome and presented via MHC-I. The MHC-antigen is recognized by a CD8⁺ T-cell. Consequently, targeting polytope processing to the cytosole skews the immune response toward a CD8⁺ response. On the other hand, Prior Art FIG. 14 illustrates a polytope predominantly processed in the endosome and presented via MHC-II. The MHC-antigen in this circumstance is recognized by a CD4⁺ T-cell. Consequently, targeting polytope processing to endosomes or lysosomes skews the immune response towards a CD4⁺ response. Such targeting methods deliver polytope and neoepitope peptides to specific MHC subtypes having the highest affinity with the peptide, even if the peptides would not otherwise present from that MHC subtype. In the examples below, further added amino acids allowed for processing flexibility in the cytoplasm, proteasome, and endosome.

Neoepitope or polytope trafficking modes may be combined to accommodate specific purposes. For example, sequential administration of the same neoepitopes or polytope with different targeting may function in a prime-boost regimen. A first administration inoculates the patient with a recombinant virus to infect patient cells, leading to antigen expression, processing, and MHC-I presentation to achieve a first immune response originating from within a cell. The second administration of the same neoepitopes bound to albumin then boosts immunity as APCs present the protein on MHC-II. Trafficking the same neoepitopes or polytope for cell surface bound MHC-independent presentation promotes ADCC responses or NK mediated cell killing. As illustrated in the examples below, cross presentation or MHC-II directed presentation can enhance neoepitope immunogenicity.

Multiple and distinct trafficking of the same neoepitopes or polytopes may be achieved in numerous manners. For example, differently trafficked neoepitopes or polytopes may be administered separately using the same (e.g., viral expression vector) or different (e.g., viral expression vector and albumin bound) modality. Similarly, and especially where the therapeutic agent is an expression system (e.g., viral or bacterial), the recombinant nucleic acid may include two distinct portions that encode the same, albeit differently trafficked neoepitope or polytope (e.g., first portion trafficked to first location (e.g., cytosol or endosomal or lysosomal), second portion trafficked to a second, distinct location (e.g., cytosol or endosomal or lysosomal, secreted, membrane bound)). Likewise, a first administration may targeted neoepitopes or polytope to the cytoplasm, while a second administration—typically at least a day, two days, four days, a week, or two weeks after the first administration—may target neoepitopes or polytope to the endosome or lysosome, or secrete them extracellularly.

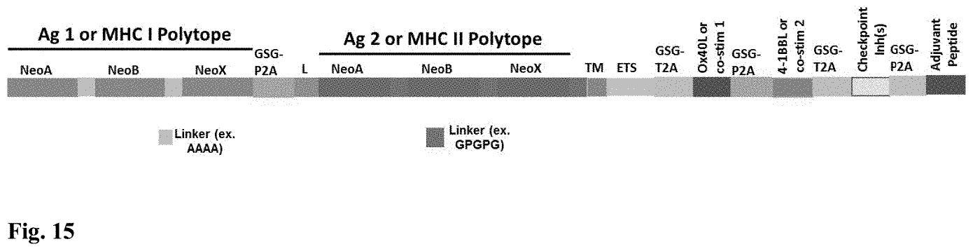

One exemplary arrangement of neoepitopes and protein adjuvant is depicted in FIG. 15. Here, the recombinant nucleic acid encodes a first series of neoepitopes coupled together by linkers. This first segment is coupled to a second series of neoepitopes coupled together by respective linkers. Between first and second segments is a GSG-P2A self-cleaving peptide sequence. Downstream of the second series of neoepitopes is a segment encoding two separate co-stimulatory molecules, followed by a segment encoding a checkpoint inhibitor. Still further downstream the checkpoint inhibitor coding sequence is a segment encoding the adjuvant peptide. The arrangement of FIG. 15 is only illustrative. Other arrangements and contents are also suitable.

While not shown in FIG. 15, human CD74-derivative sequence elements—“Ii-keys”—included in the recombinant nucleic acid can increase MHC-II presented epitope immunogenicity. Exemplary sequence elements include “LRMKLPKPPKPVSKIVIR” as well as shorter versions thereof, especially “LRMK”. Such sequence elements may be placed 5′ of the patient and/or tumor specific neoepitope(s). For example, constructs can contain one or more “Ii-key” sequences in an MHC II targeting polytope, either immediately after the leader peptide sequence and prior to the polytope sequence, optionally with one or more intra-epitope linkers (GPGPG-LRMK) to augment each epitope.

The expression construct (e.g., expression vector or plasmid) may further encode at least one, at least two, at least three, or even at least four co-stimulatory molecules to enhance interaction between the infected cells (e.g., APCs) and T-cells. Non-limiting examples include CD80, CD86, CD30, CD40, CD30L, CD40L, ICOS-L, B7-H3, B7-H4, CD70, OX40L, 4-1BBL, or even GITR-L, TIM-3, TIM-4, CD48, CD58, TL1A, ICAM-1, LFA3, and members of the SLAM family. Especially preferred molecules for coordinated expression include CD80 (B7-1), CD86 (B7-2), CD54 (ICAM-1) and CD11 (LFA-1). One or more cytokines or cytokine analogs may also be expressed from the recombinant nucleic acid. Non-limiting examples include IL-2, IL-15, and IL-15 superagonist (ALT-803). Expression of the co-stimulatory molecules and/or cytokines can be coordinated such that neoepitopes or polytopes are expressed contemporaneously with the co-stimulatory molecules and/or cytokines. The co-stimulatory molecules and/or cytokines can be produced from a single transcript (optionally including the polytope coding sequence), for example, using an IRES or 2A sequence, or from multiple transcripts.

The viral vector may also encode one or more checkpoint receptor ligands. Most typically, binding inhibits checkpoint signaling. Non-limiting receptor examples include CTLA-4 (especially for CD8⁺ cells), PD-1 (especially for CD4⁺ cells), TIM1 receptor, 2B4, and CD160. Suitable peptide binders can include antibody fragments, especially scFv. Small molecule peptide ligands (e.g., isolated via RNA display or phage panning) that specifically bind receptors are also useful. Expression of the checkpoint inhibitors can be coordinated such that the neoepitopes or polytope are expressed contemporaneously. Ligands can be produced from a single transcript (optionally including the polytope coding sequence), for example, using an IRES or 2A sequence, or from multiple transcripts.

All of the above noted co-stimulators and checkpoint inhibitors are well known in the art, and sequence information for genes encoding these proteins, isoforms, and variants can be retrieved from various public resources, including sequence databases accessible at the NCBI, EMBL, GenBank, RefSeq, etc. While the above exemplary stimulating molecules can be expressed in full length, human form, modified and non-human forms are also suitable so long as such forms stimulate or activate T-cells. Therefore, muteins, truncations and chimeras are also suitable.

Expression constructs preferably include a sequence encoding one or more polytopes, wherein at least one, at least two, or all the polytopes include trafficking signals that direct the polytope to at least one, and more typically at least two sub-cellular locations. For example, the first polytope may traffic to the cytoplasm while the second traffics to the endosome or lysosome. Or the first polytope may traffic to the endosome or lysosome while the second traffics to the cell membrane or secretion.

Viral expression constructs (e.g., adenovirus, especially ΔE1/ΔE2b AdV5) may be used individually or in combination as therapeutic vaccines in treatments accompanied by allografted or autologous natural killer cells, or T cells—in a bare form or bearing chimeric antigen receptors expressing antibodies targeting neoepitope, neoepitopes, tumor associated antigens or the same payload as the virus. The natural killer cells, which include the patient-derived NK-92 cell line, may also express CD16, and can be coupled with an antibody.

Additional therapeutic neoepitope based modalities (e.g., synthetic antibodies against neoepitopes as described in WO 2016/172722) may be administered, alone or in combination with autologous or allogenic NK cells, and especially haNK cells or taNK cells (e.g., both commercially available from NantKwest, 9920 Jefferson Blvd. Culver City, Calif. 90232). By way of non-limiting example, haNK cells may carry a recombinant antibody on the CD16 variant that binds to a neoepitope of the treated patient, and taNK cells may carry a chimeric antigen receptor that binds to a neoepitope of the treated patient. The additional treatment modality may also be independent of neoepitopes, such as activated NK cells (e.g., aNK cells, commercially available from NantKwest, 9920 Jefferson Blvd. Culver City, Calif. 90232), and non cell-based therapeutics such as chemotherapy and/or radiation. Immune stimulatory cytokines—especially IL-2, IL15, & IL-21—may be administered, alone or in combination with one or more checkpoint inhibitors (e.g., ipilimumab, nivolumab, etc.). Additional pharmaceutical intervention may include administration of one or more drugs that inhibit immune suppressive cells, especially MDSCs, Tregs, and M2 macrophages. Suitable drugs for this purpose include IL-8 or interferon-γ inhibitors, or antibodies binding IL-8 or interferon-γ, as well as drugs that deactivate MDSCs (e.g., NO inhibitors, arginase inhibitors, ROS inhibitors), that block development of or differentiation to MDSCs (e.g., IL-12, VEGF-inhibitors, bisphosphonates), or agents toxic to MDSCs (e.g., gemcitabine, cisplatin, 5-FU). Likewise, cyclophosphamide, daclizumab, and anti-GITR or anti-OX40 antibodies can inhibit Tregs.

Chemotherapy and/or radiation at low-dose, preferably in a metronomic regimen can trigger overexpression or transcription of stress signals. Such treatment can use doses that affect at protein expression, cell division, and/or cell cycle, preferably to induce apoptosis or stress-related genes (particularly NKG2D ligands). Such treatments may include low dose treatment using one or more chemotherapeutics. Most typically, low dose treatments exposures should be no more than 70%, equal or less than 50%, equal or less than 40%, equal or less than 30%, equal or less than 20% , equal or less than 10%, or equal or less than 5% of the LD₅₀ or IC₅₀ for the chemotherapeutic. Such low-dose regimen may be performed in metronomically as described in U.S. Pat. Nos. 7,758,891, 7,771,751, 7,780,984, 7,981,445, and 8,034,375.

All known chemotherapeutis are suitable for use in the methods disclosed herein, including by way of non-limiting example kinase inhibitors, receptor agonists & antagonists, anti-metabolic, cytostatic, and cytotoxic drugs. Drugs suitable to interfere or inhibit a pathway that drives tumor growth or development can be identified using pathway analysis on omics data as described in, WO 11/139345 and WO 13/62505. Expression of stress-related genes in tumor cells drives surface presentation of NKG2D, NKP30, NKP44, and/or NKP46 ligands, which activate NK cells to destroy tumor cells. Low-dose chemotherapy can trigger tumor cells to express and display stress related proteins.

While numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

Unless the context dictates the contrary, all ranges set forth herein should be interpreted as being inclusive of their endpoints and open-ended ranges should be interpreted to include only commercially practical values. Similarly, all lists of values should be considered as inclusive of intermediate values unless the context indicates the contrary. As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

It should be apparent to those skilled in the art that many more modifications besides those already described are possible without departing from the concepts disclosed herein. The claimed subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . . and N, the text should be interpreted as requiring only one element from the group, not A plus N, or B plus N, etc.

1. An expression cassette comprising a promoter operably coupled to a recombinant nucleic acid having first and second nucleic acid segments;
wherein the first nucleic acid segment encodes a chimeric protein having an extracellular portion of a TNF family ligand coupled by a flexible linker to its corresponding TNF family receptor; and
wherein the second nucleic acid segment encodes a tumor-associated antigen (TAA).

2. The expression cassette of claim 1, wherein the extracellular portion of the TNF family ligand is nearer the N-terminus than is the corresponding TNF family receptor on the chimeric protein.

3. The expression cassette of claim 2, wherein the TNF family ligand is CD40L and wherein the TNF family receptor is CD40.

4. The expression cassette of claim 3, wherein the recombinant nucleic acid further comprises a third nucleic acid segment encoding a leader peptide coupled to the N-terminus of the extracellular portion of CD40L.

5. The expression cassette of claim 3, wherein the extracellular portion of CD40L is a human extracellular portion of CD40L.

6. The expression cassette of claim 5, wherein the flexible linker has between 4 and 50 amino acids, and comprises a (GnS)x sequence.

7. The expression cassette of claim 3, wherein the TAA is selected from the group consisting of brachyury, MUC1, and CEA.

8. (canceled)

9. The expression cassette of claim 3, wherein the first and second nucleic acid segments are placed in the same reading frame.

10. The expression cassette of claim 3, wherein the first and second nucleic acid segments are coupled via an IRES sequence.

11. The expression cassette of claim 3, wherein the first and second nucleic acid segments are separated by a 2A sequence.

12. The expression cassette of claim 11, further comprising a fourth nucleic acid segment encoding an immune stimulatory cytokine, wherein the immune stimulatory cytokine is an IL-15 super agonist (ALT803) coupled with at least one of IL-7 and IL-21.

13. (canceled)

14. (canceled)

15. A human adenovirus type 5 (AdV5) [E1-, E2b-] comprising the expression cassette of claim 3.

16. A method of treating a patient having a tumor, the method comprising administering to the patient the adenovirus of claim 15.

17. The method of claim 16, further comprising administering to the patient a checkpoint inhibitor.

18. (canceled)

19. The method of claim 16, further comprising co-administering a genetically modified bacteria or a genetically modified yeast as an adjuvant to the virus.

20. The method of claim 19, wherein the genetically modified bacteria expresses endotoxins at a level insufficient to induce CD-14 mediated sepsis.

21. The method of claim 19, wherein the genetically modified yeast belongs to a GI-400 series recombinant immunotherapeutic yeast strain.

22. A genetically modified dendritic cell comprising an expression cassette according to claim 3.

23-25. (canceled)

26. A method of generating an expression vector for enhanced immune therapy, the method comprising:
using matched normal omics data of a tumor to generate in silico a plurality of n-mers that contain at least one patient- and cancer-specific cancer neoepitope wherein the omics data from each of the tumor and the matched patient normal sample include data selected from the group consisting of whole genomic sequencing data, exome sequencing data, transcriptome data, and combinations thereof;
filtering in silico the n-mers to so obtain a subset of neoepitope sequences wherein the filtering is filtering by type of mutation, filtering by strength of expression, filtering by subcellular location, and/or filtering by binding affinity towards an HLA-type of the patient;
constructing a recombinant nucleic acid having a sequence that encodes (a) a polytope operably linked to a first promoter to drive expression of the polytope, and (b) an adjuvant polypeptide operably linked to a second promoter to drive expression of the adjuvant polypeptide;
wherein the polytope comprises a plurality of the filtered neoepitopes and a trafficking element that directs the polytope to a sub-cellular location selected from the group consisting of cytoplasm, recycling endosome, sorting endosome, lysosome, and extracellular membrane, or
wherein the trafficking element directs the polytope to an extracellular space; and wherein the polytope comprises a plurality of filtered neoepitope sequences.

27-44. (canceled)

45. A method of improving an immune response to cancer immune therapy in an individual with a tumor, the method comprising:
administering to the tumor a cancer vaccine composition; and
co-administering to the tumor at substantially the same time an adjuvant polypeptide, ATP, or an ATP analog.

46-61. (canceled)
